(19) United States
(12) Patent Application Publication
(10) Pub. No.: US 2022/0357449 A1
(43) Pub. Date: Nov. 10, 2022

(54) INTRAORAL MEASUREMENT DEVICE AND INTRAORAL MEASUREMENT SYSTEM

(71) Applicant: Murata Manufacturing Co., Ltd., Kyoto (JP)

(72) Inventors: Tomoki TAKAHASHI, Kyoto (JP); Jun TAKAGI, Kyoto (JP); Hiroaki TOGASHI, Kyoto (JP)

(21) Appl. No.: 17/813,661

(22) Filed: Jul. 20, 2022

Related U.S. Application Data
(63) Continuation of application No. PCT/JP2020/038648, filed on Oct. 13, 2020.

(30) Foreign Application Priority Data
Jan. 30, 2020 (JP) 2020-013614

(51) Int. Cl. G01S 17/42; G01S 7/481

(52) U.S. Cl. CPC G01S 17/42; G01S 7/4811

(57) ABSTRACT

An intraoral measurement device according to the present disclosure is an intraoral measurement device having a contact surface that comes into contact with a measurement site in an oral cavity, the device includes: a biological sensor that is arranged on the contact surface, and has a detection surface that acquires biological information; and one or a plurality of contact detection units that are arranged at least either on the biological sensor or at a periphery of the biological sensor, and acquire contact information indicating a degree of contact between the measurement site and the contact surface.

### CROSS REFERENCE TO RELATED APPLICATION

This is a continuation of International Application No. PCT/JP2020/038648 filed on Oct. 13, 2020 which claims priority from Japanese Patent Application No. 2020-013614 filed on Jan. 30, 2020. The contents of these applications are incorporated herein by reference in their entireties.

### BACKGROUND ART

### Technical Field

The present disclosure relates to an intraoral measurement device and an intraoral measurement system for measuring an inside of an oral cavity.

Patent Document 1 discloses an intraoral moisture measuring device. The intraoral moisture measuring device disclosed in Patent Document 1 includes a swing member, a moisture amount detection unit provided at a distal end of the swing member, and a biasing member that biases the swing member in one of swing directions.

Patent Document 1: WO 2015/125222 A

### BRIEF SUMMARY

In recent years, there has been a demand for an intraoral measurement device and an intraoral measurement system with improved measurement accuracy.

An intraoral measurement device according to one aspect of the present disclosure is an intraoral measurement device having a contact surface that comes into contact with a measurement site in an oral cavity, the device including:

a biological sensor that is arranged on the contact surface, and has a detection surface that acquires biological information; and

one or a plurality of contact detection units that are arranged at least either on the biological sensor or at a periphery of the biological sensor, and acquire contact information indicating a degree of contact between the measurement site and the contact surface.

According to the present disclosure, it is possible to provide an intraoral measurement device and an intraoral measurement system with improved measurement accuracy.

### BRIEF DESCRIPTION OF THE DRAWINGS

FIG. 1 is a schematic perspective view of an example of an intraoral measurement device of a first embodiment according to the present disclosure.

FIG. 2 is a schematic view illustrating an internal configuration of the example of the intraoral measurement device of the first embodiment according to the present disclosure.

FIG. 3 is a block diagram illustrating a schematic configuration of the example of the intraoral measurement device of the first embodiment according to the present disclosure.

FIG. 4 is a schematic enlarged sectional view of an example of a sensor unit in the intraoral measurement device of the first embodiment according to the present disclosure.

FIG. 5 is a schematic enlarged bottom view of the example of the sensor unit in the intraoral measurement device of the first embodiment according to the present disclosure.

FIG. 6 is a flowchart illustrating an example of an operation of the intraoral measurement device of the first embodiment according to the present disclosure.

FIG. 7 is a flowchart illustrating an example of contact detection processing.

FIG. 8 is a schematic view illustrating an example of the contact detection processing.

FIG. 9 is a schematic view illustrating an example of the contact detection processing.

FIG. 10 is a schematic view illustrating an example of the contact detection processing.

FIG. 11 is a schematic view illustrating an example of a mode in which the intraoral measurement device of the first embodiment according to the present disclosure is used.

FIG. 12 is a schematic perspective view of an example of an intraoral measurement device of a second embodiment according to the present disclosure.

FIG. 13 is a flowchart illustrating an example of an operation of the intraoral measurement device of the second embodiment according to the present disclosure.

FIG. 14 is a block diagram illustrating a schematic configuration of an example of an intraoral measurement device of a third embodiment according to the present disclosure.

FIG. 15 is a flowchart illustrating an example of an operation of the intraoral measurement device of the third embodiment according to the present disclosure.

FIG. 16 is a block diagram illustrating a schematic configuration of an example of an intraoral measurement system of a fourth embodiment according to the present disclosure.

FIG. 17 is a flowchart illustrating an example of an operation of the intraoral measurement system of the fourth embodiment according to the present disclosure.

FIG. 18 is a schematic enlarged view of an example of an intraoral measurement device of a fifth embodiment according to the present disclosure.

FIG. 19 is a schematic enlarged view of an example of an intraoral measurement device of a sixth embodiment according to the present disclosure.

FIG. 20 is a schematic enlarged view of an intraoral measurement device of a modification of the sixth embodiment according to the present disclosure.

FIG. 21 is a schematic enlarged view of an intraoral measurement device of a seventh embodiment according to the present disclosure.

FIG. 22 is a schematic enlarged view of an intraoral measurement device of a modification of the seventh embodiment according to the present disclosure.

FIG. 23 is a schematic enlarged view of an example of an intraoral measurement device of an eighth embodiment according to the present disclosure.

FIG. 24 is a schematic enlarged view of an example of an intraoral measurement device of a ninth embodiment according to the present disclosure.

FIG. 25A is a schematic view of an example of an operation of the intraoral measurement device of the ninth embodiment according to the present disclosure.

FIG. 25B is a schematic view of an example of an operation of the intraoral measurement device of the ninth embodiment according to the present disclosure.

FIG. 25C is a schematic view of an example of an operation of the intraoral measurement device of the ninth embodiment according to the present disclosure.

### DETAILED DESCRIPTION

### (Circumstances Leading to Present Disclosure)

As an intraoral measurement device, an intraoral moisture measuring device described in Patent Document 1 is known, for example. When measuring a moisture amount in an oral cavity, the intraoral moisture measuring device described in Patent Document 1 detects a pressing force of the moisture amount detection unit against a measurement site, and starts measurement of the moisture amount on the basis of the pressing force.

However, there is a problem that the measurement accuracy of the moisture amount is lowered in a case where the contact between a detection surface of a sensor and a measurement site is insufficient as in the device described in Patent Document 1. In addition, there is a problem that it is difficult for a user to visually check when the detection surface of the sensor is brought into contact with the measurement site in the oral cavity.

Therefore, inventors of the present disclosure have found a configuration in which a biological sensor having a detection surface and a contact detection unit are provided, and the contact between a measurement site and the detection surface of the biological sensor is detected with high accuracy by devising the position of the contact detection unit, and have reached the following disclosure.

An intraoral measurement device according to one aspect of the present disclosure is an intraoral measurement device having a contact surface that comes into contact with a measurement site in an oral cavity, the device including:

a biological sensor that is arranged on the contact surface, and has a detection surface that acquires biological information; and

one or a plurality of contact detection units that are arranged at least either on the biological sensor or at a periphery of the biological sensor, and acquire contact information indicating a degree of contact between the measurement site and the contact surface.

With such a configuration, the measurement accuracy can be improved.

The intraoral measurement device may include

a housing that has a rod shape and houses the biological sensor and the contact detection units, and

the contact surface may be provided on one end side in a longitudinal direction of the housing, and be provided in a direction intersecting an end surface on the one end side.

With such a configuration, usability for the user is improved.

The one or the plurality of contact detection units may be arranged at positions farther from one end in the longitudinal direction of the housing than the biological sensor.

With such a configuration, the measurement accuracy can be further improved.

The one or the plurality of contact detection units may be arranged at positions closer to one end in the longitudinal direction of the housing than the biological sensor.

With such a configuration, the measurement accuracy can be further improved.

The plurality of contact detection units may include:

one or a plurality of first contact detection units arranged at positions closer to one end in the longitudinal direction of the housing than the biological sensor; and one or a plurality of second contact detection units arranged at positions farther from the one end in the longitudinal direction of the housing than the biological sensor.

With such a configuration, the measurement accuracy can be further improved.

The detection surface of the biological sensor may have a polygonal shape, and

the plurality of contact detection units may be arranged at corners of the detection surface.

With such a configuration, the measurement accuracy can be further improved.

The plurality of contact detection units may be arranged symmetrically about the biological sensor.

With such a configuration, the measurement accuracy can be further improved.

The one or the plurality of contact detection units may surround a periphery of the detection surface of the biological sensor.

With such a configuration, the measurement accuracy can be further improved.

The one or the plurality of contact detection units may include:

one or a plurality of optical sensors that receive light; and

one or a plurality of light guide units that are arranged on the contact surface and guide the light to the optical sensors.

With such a configuration, the measurement accuracy can be further improved.

Each of the optical sensors may include:

a light emitting unit that emits light; and

a light receiving unit that receives light reflected by the measurement site.

With such a configuration, the measurement accuracy can be further improved.

The biological sensor may have translucency and have an arrangement surface on a side opposite to the detection surface, and

the one or the plurality of contact detection units may have one or a plurality of optical sensors that receive light, and be arranged on the arrangement surface of the biological sensor.

Such a configuration can reduce the number of components, so that miniaturization of the device can be realized.

The intraoral measurement device may further include a light emitting unit that is arranged on the contact surface and emits light.

With such a configuration, the measurement accuracy can be further improved.

The biological sensor is a capacitive sensor that detects a capacitance.

With such a configuration, a capacitance can be acquired as biological information.

The intraoral measurement device may further include a processing unit that outputs trigger information for starting processing of calculating an amount of a measurement object on a basis of an output value of the biological sensor and output values of the contact detection units.

With such a configuration, the measurement accuracy can be further improved.

Hereinafter, embodiments of the present disclosure will be described with reference to the accompanying drawings. Note that the following description is merely exemplary in nature, and is not intended to limit the present disclosure, its application, or its use. Furthermore, the drawings are schematic, and ratios of dimensions and the like do not necessarily match actual ones.

### (First Embodiment)

### [Overall Configuration]

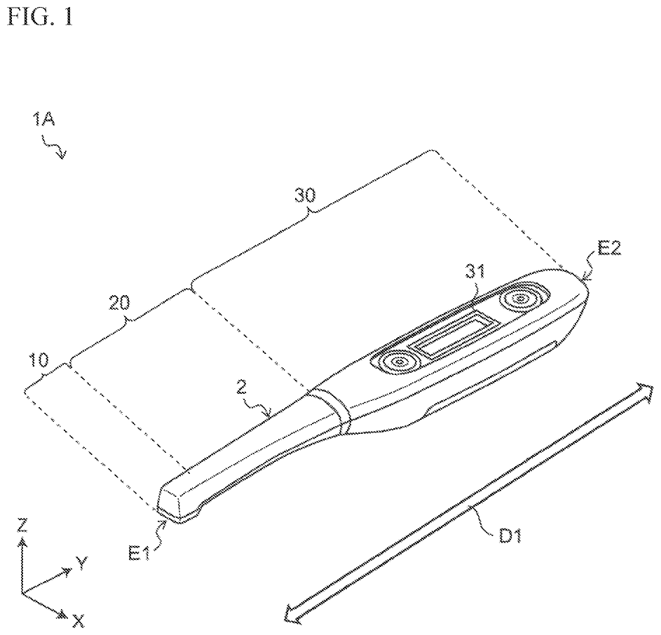

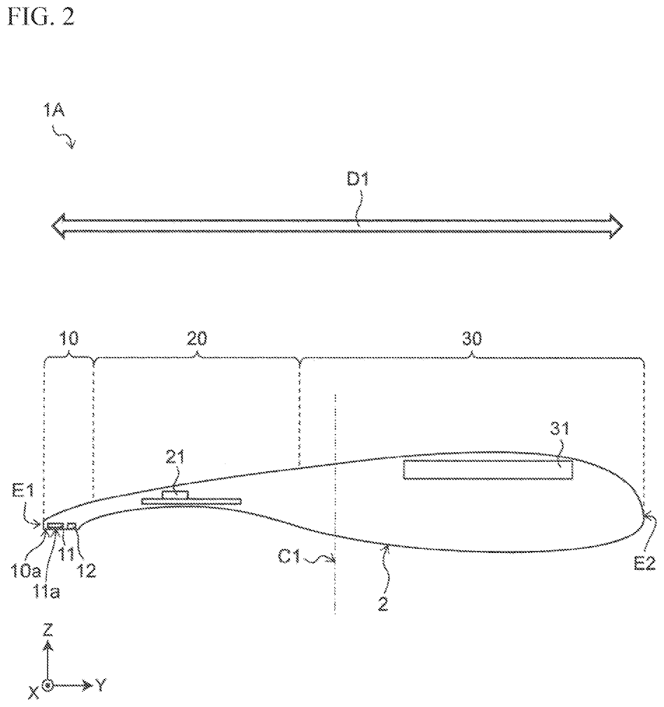

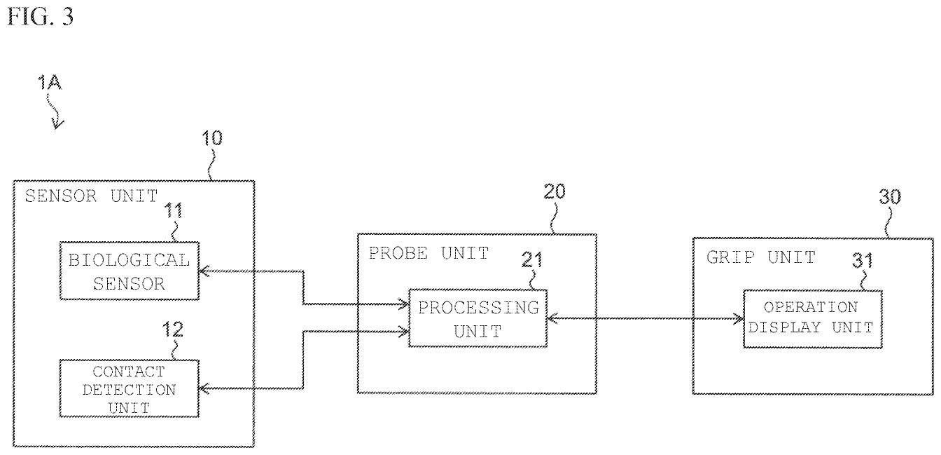

FIG. 1 is a schematic perspective view of an example of an intraoral measurement device 1A of a first embodiment according to the present disclosure. FIG. 2 is a schematic view illustrating an internal configuration of the example of the intraoral measurement device 1A of the first embodiment according to the present disclosure. FIG. 3 is a block diagram illustrating a schematic configuration of the example of the intraoral measurement device 1A of the first embodiment according to the present disclosure. In the drawings, X, Y, and Z directions respectively indicate a width direction, a length direction, and a height direction of the intraoral measurement device 1A.

### <Appearance>

The appearance of the intraoral measurement device 1A will be described. As illustrated in FIGS. 1 and 2, the intraoral measurement device 1A includes a housing 2. The housing 2 has a rod-like shape and extends in a longitudinal direction D1. Specifically, the housing 2 includes a sensor unit 10, a probe unit 20, and a grip unit 30.

The sensor unit 10 is a portion that comes into contact with a measurement site in an oral cavity of a user. The measurement site in the oral cavity is, for example, a tongue portion. The sensor unit 10 is provided at one end E1 in the longitudinal direction D1 of the intraoral measurement device 1A. Outer dimensions of the sensor unit 10 are designed to be smaller than those of the probe unit 20 and the grip unit 30. For example, dimensions of the sensor unit 10 in the X direction and in the Y direction are designed to be small compared to those of the probe unit 20 and the grip unit 30.

The sensor unit 10 has a contact surface 10a that comes into contact with the measurement site in an oral cavity of a user. The contact surface 10a is provided at one end E1 side in the longitudinal direction D1 of the housing 2, and is provided in a direction (X, Y direction) intersecting the end surface on the one end E1 side.

The probe unit 20 connects the sensor unit 10 and the grip unit 30. The probe unit 20 is formed in a rod shape. In the probe unit 20, dimensions in the X direction and in the Z direction decrease from the grip unit 30 toward the sensor unit 10. That is, the probe unit 20 has a shape tapered from the grip unit 30 toward the sensor unit 10.

The grip unit 30 is a portion arranged outside the oral cavity of a user and gripped by the user. The grip unit 30 is provided at the other end E2 in the longitudinal direction D1 of the intraoral measurement device 1A. The grip unit 30 is formed in a rod shape. Outer dimensions of the grip unit 30 are designed to be larger than those of the sensor unit 10 and the probe unit 20. For example, dimensions of the grip unit 30 in the X, Y, and Z directions are designed to be large compared to those of the sensor unit 10 and the probe unit 20.

The housing 2 is made of, for example, resin. In addition, a part of the housing 2 may be made of metal. Alternatively, the entire housing 2 may be made of metal.

Next, constituent elements that constitute the intraoral measurement device 1A will be described. As illustrated in FIGS. 1 to 3, the intraoral measurement device 1A includes a biological sensor 11, a contact detection unit 12, a processing unit 21, and an operation display unit 31.

Note that, in the first embodiment, an example will be described in which the intraoral measurement device 1A is provided with the operation display unit 31, but the present disclosure is not limited thereto. The operation display unit 31 is not an essential component, and may be provided in a device different from the intraoral measurement device 1A.

Further, in the first embodiment, an example will be described in which a measurement object of the intraoral measurement device 1A is moisture, and a moisture amount is measured using the intraoral measurement device 1A.

### <Biological Sensor>

The biological sensor 11 acquires biological information. The biological information is various types of physiological and anatomical information generated by a living body. The biological information is, for example, information on a capacitance, a resistance value, a moisture amount, a temperature, hardness, and the like. The biological sensor 11 comes into contact with a measurement site in an oral cavity of a user, and acquires the biological information of the contacted measurement site.

In the first embodiment, the biological sensor 11 is, for example, a capacitive sensor. The biological sensor 11 comes into contact with the measurement site in an oral cavity, and acquires information on a capacitance. That is, in the first embodiment, the biological information acquired by the biological sensor 11 is information on a capacitance.

The biological sensor 11 is arranged on the contact surface 10a. For example, the biological sensor 11 is arranged in a recess formed in a contact surface 10a side of the sensor unit 10 of the housing 2.

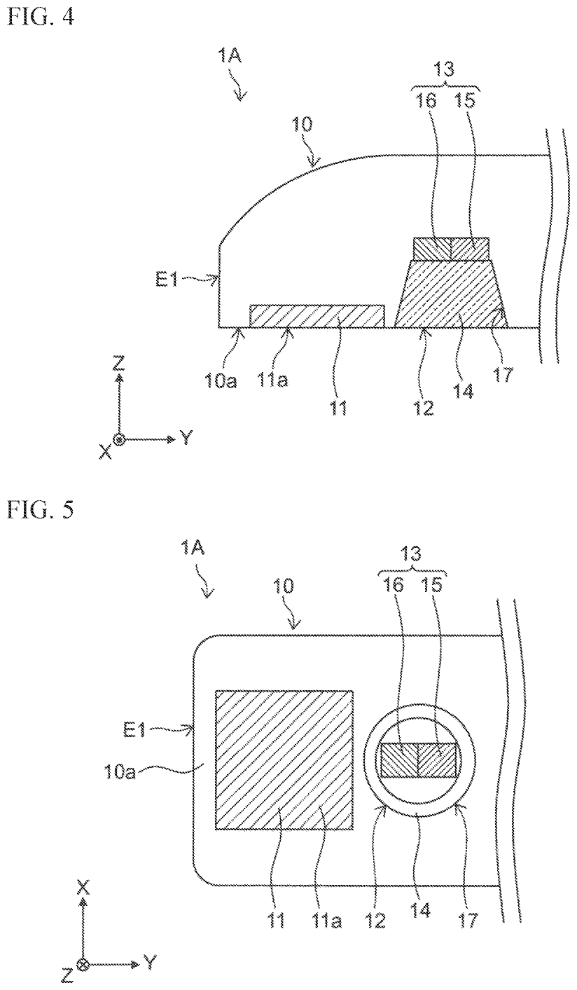

FIG. 4 is a schematic enlarged sectional view of an example of the sensor unit 10 in the intraoral measurement device 1A of the first embodiment according to the present disclosure. FIG. 5 is a schematic enlarged bottom view of the example of the sensor unit 10 in the intraoral measurement device 1A of the first embodiment according to the present disclosure. As illustrated in FIGS. 4 and 5, the biological sensor 11 is arranged on the contact surface 10a on the one end E1 side in the longitudinal direction D1 of the intraoral measurement device 1A.

The biological sensor 11 is formed in a planar shape. Specifically, the biological sensor 11 has a detection surface 11a that acquires biological information. The detection surface 11a is exposed to the contact surface 10a side of the sensor unit 10. For example, the detection surface 11a is formed in a rectangular shape as viewed in the height direction (Z direction) of the intraoral measurement device 1A. The detection surface 11a detects the biological information by coming into contact with the measurement site. That is, the biological sensor 11 acquires the biological information by bringing the detection surface 11a into contact with the measurement site.

The biological information acquired by the biological sensor 11 is transmitted to the processing unit 21.

### <Contact Detection Unit>

The contact detection unit 12 acquires contact information between the measurement site in an oral cavity and the contact surface 10a. The contact information indicates a degree of contact between the measurement site in the oral cavity and the contact surface 10a. The degree of contact relates to, for example, the distance and/or the contact area between the measurement site and the contact surface 10a.

The contact detection unit 12 is housed inside the sensor unit 10 of the housing 2. Specifically, the contact detection unit 12 is arranged on the contact surface 10a at the periphery of the biological sensor 11. That is, the contact detection unit 12 is arranged at the periphery of the detection surface 11a of the biological sensor 11. This makes it easier to detect whether or not the detection surface 11a of the biological sensor 11 is in contact with the measurement site.

In the first embodiment, the contact detection unit 12 is arranged at a position farther from the one end E1 in the longitudinal direction D1 of the housing 2 than the biological sensor 11. In other words, the contact detection unit 12 is arranged on a center C1 side of the housing 2 with respect to the biological sensor 11 in the longitudinal direction D1 of the intraoral measurement device 1A. In addition, the contact detection unit 12 is arranged at the center in the width direction (X direction) of the intraoral measurement device 1A as viewed in the height direction (Z direction) of the intraoral measurement device 1A.

In the first embodiment, the contact detection unit 12 includes an optical sensor 13 and a light guide unit 14.

The optical sensor 13 is a sensor that receives light. The optical sensor 13 is, for example, a light sensor, an infrared sensor, or a laser distance sensor. In the first embodiment, the optical sensor 13 is a light sensor that receives light guided by the light guide unit 14 and detects photovoltaic power. In the first embodiment, the contact information is information on photovoltaic power.

The optical sensor 13 includes a light emitting unit 15 that emits light and a light receiving unit 16 that receives light reflected by the measurement site. For example, the light emitting unit 15 is constituted of an LED. The light receiving unit 16 is constituted of a photodiode.

The optical sensor 13 is arranged inside the housing 2. Specifically, the optical sensor 13 is arranged on a recessed surface of a recess 17 formed in the contact surface 10a of the sensor unit 10. The recess 17 is a hole recessed from the contact surface 10a in the height direction (Z direction) of the intraoral measurement device 1A. The recessed surface is provided inside the sensor unit 10 with respect to the contact surface 10a. In the first embodiment, the recess 17 is formed in a truncated cone shape, the diameter of which increases from the inside of the sensor unit 10 toward the contact surface 10a in the height direction (Z direction) of the intraoral measurement device 1A.

The light guide unit 14 is provided on the contact surface 10a and guides light to the optical sensor 13. For example, the light guide unit 14 is a light guide plate or a lens. The light guide unit 14 is arranged in the recess 17. The light guide unit 14 is arranged in contact with a light emitting surface of the light emitting unit 15 and a light receiving surface of the light receiving unit 16. In other words, the light guide unit 14 is arranged between the contact surface 10a, and the light emitting surface of the light emitting unit 15 and the light receiving surface of the light receiving unit 16.

The contact information acquired by the contact detection unit 12 is transmitted to the processing unit 21.

### <Processing Unit>

The processing unit 21 outputs trigger information for starting processing of calculating the amount of the measurement object on the basis of an output value of the biological sensor 11 and an output value of the contact detection unit 12.

The processing of calculating the amount of the measurement object is performed by a calculation unit. The calculation unit may be provided in the intraoral measurement device 1A, or be provided in a device different from the intraoral measurement device 1A. The processing unit 21 transmits the trigger information to the calculation unit. The calculation unit starts the processing of calculating the amount of the measurement object on the basis of the trigger information.

The processing unit 21 is arranged on a biological sensor 11 side in the longitudinal direction D1 of the intraoral measurement device 1A with respect to the center C1. Specifically, the processing unit 21 is arranged inside the probe unit 20. This can suppress generation of noise.

The processing unit 21 includes a frequency conversion circuit that converts information on a capacitance, which is biological information acquired by the biological sensor 11, into a frequency. The processing unit 21 receives information on a capacitance from the biological sensor 11, and converts the capacitance into a frequency by the frequency conversion circuit.

For example, the processing unit 21 repeatedly performs charging and discharging with respect to the biological sensor 11 regarded as the capacitance, and converts the capacitance into a frequency of a cycle determined by the charging and discharging speed.

In the first embodiment, the output value of the biological sensor 11 is a frequency converted from the capacitance by the frequency conversion circuit of the processing unit 21. The output value of the contact detection unit 12 is photovoltaic power detected by the light sensor.

The processing unit 21 outputs trigger information on the basis of a first threshold value S1 of the output value of the contact detection unit 12 and a second threshold value S2 of the output value of the biological sensor 11. For example, the processing unit 21 outputs the trigger information when the output value of the contact detection unit 12 is equal to or less than the first threshold value S1, and the output value of the biological sensor 11 is equal to or less than the second threshold value S2. The determination based on the first threshold value S1 and the second threshold value S2 may be changed according to the types of the output of the biological sensor 11 and the output of the contact detection unit 12. For example, the processing unit 21 may output the trigger information when the output value of the contact detection unit 12 is greater than or equal to the first threshold value S1, and the output value of the biological sensor 11 is greater than or equal to the second threshold value S2.

Alternatively, the processing unit 21 may output the trigger information on the basis of the variation range of the output value of the contact detection unit 12 and the variation range of the output value of the biological sensor 11. In this case, a threshold value of the variation range of the output value of the contact detection unit 12 and a threshold value of the variation range of the output value of the biological sensor 11 may be set. That is, the processing unit 21 may output the trigger information on the basis of the threshold value of the variation range of the output value of the contact detection unit 12 and the threshold value of the variation range of the output value of the biological sensor 11. For example, the processing unit 21 may output the trigger information when both the variation range of the output value of the contact detection unit 12 and the variation range of the output value of the biological sensor 11 exceed the threshold values. As a result, the contact between the measurement site and the contact surface 10a can be detected with high accuracy without necessarily being affected by the individual difference between users.

The processing unit 21 can be implemented by a semiconductor element or the like. The processing unit 21 may be constituted of, for example, a microcomputer, a CPU, an MPU, a GPU, a DSP, an FPGA, an ASIC, a discrete semiconductor, and an LSI. The function of the processing unit 21 may be configured only by hardware, or be implemented by configured hardware and software. The processing unit 21 implements the predetermined function by reading data and a program stored in a storage unit (not illustrated) in the processing unit 21, and performing various types of arithmetic processing. The storage unit can be implemented by, for example, a hard disk (HDD), an SSD, a RAM, a DRAM, a ferroelectric memory, a flash memory, a magnetic disk, or combination thereof.

The processing unit 21 transmits the trigger information to the calculation unit. For example, when receiving the trigger information, the calculation unit calculates the amount of the measurement object on the basis of the output value of the biological sensor 11. In the first embodiment, the amount of the measurement object is a moisture amount. The calculation unit calculates the moisture amount on the basis of the output value of the biological sensor 11, that is, the value of the frequency.

### <Operation Display Unit>

The operation display unit 31 receives input from a user, and displays information on the amount of the measurement object. For example, the operation display unit 31 includes an operation unit that receives an operation from a user, and a display unit that displays information.

The operation unit includes one or a plurality of buttons that receive input from a user. The plurality of buttons include, for example, a power button for switching power on/off, and the like.

The display unit displays information on the amount of the measurement object. The display unit is, for example, a display. The information on the amount of the measurement object is transmitted, for example, from the calculation unit provided in the intraoral measurement device 1A to the display unit. Alternatively, the information on the amount of the measurement object is transmitted from the calculation unit provided in a device different from the intraoral measurement device 1A to the display unit via, for example, a network or the like.

The operation display unit 31 is arranged on an upper surface of the grip unit 30.

The intraoral measurement device 1A includes a control unit that comprehensively controls constituent elements constituting the intraoral measurement device 1A. The control unit includes, for example, a memory that stores a program and a processing circuit that corresponds to a processor such as a central processing unit (CPU). For example, in the control unit, the processor executes the program stored in the memory. In the first embodiment, the control unit controls the biological sensor 11, the contact detection unit 12, the processing unit 21, and the operation display unit 31.

### [Operation of Intraoral Measurement Device]

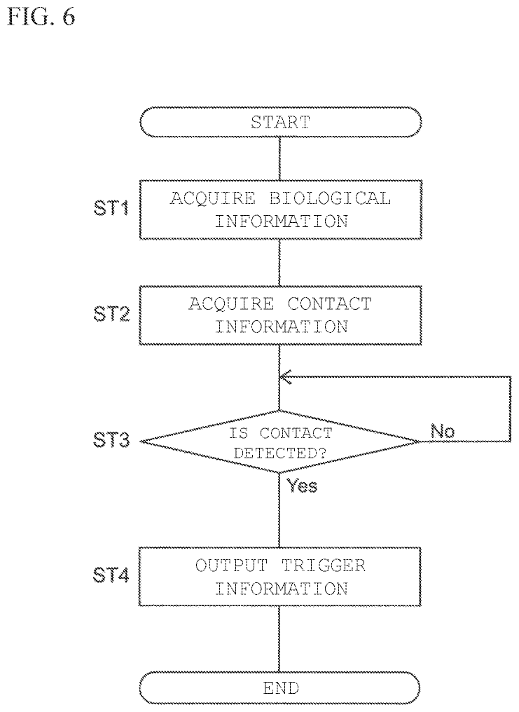

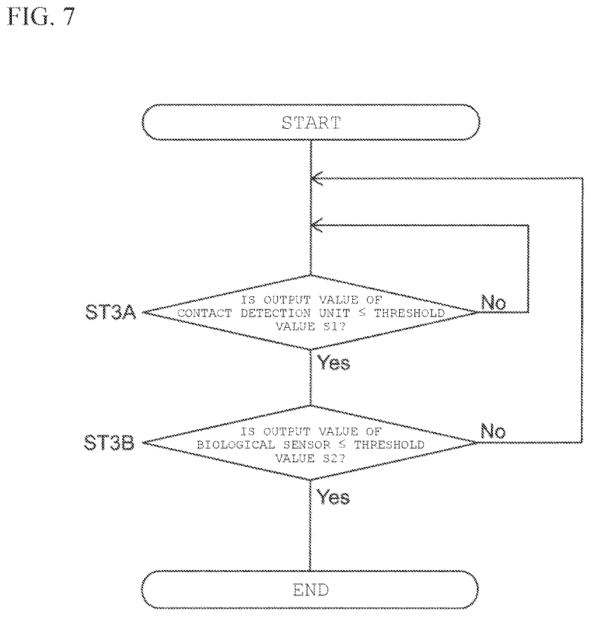

An example of an operation of the intraoral measurement device 1A, that is, an example of an intraoral measurement method will be described. FIG. 6 is a flowchart illustrating an example of an operation of the intraoral measurement device 1A of the first embodiment according to the present disclosure. FIG. 7 is a flowchart illustrating an example of contact detection processing.

As illustrated in FIG. 6, in step ST1, the biological sensor 11 acquires biological information. The biological information acquired by the biological sensor 11 is transmitted to the processing unit 21.

In the first embodiment, step ST1 is started by turning on the power at the operation display unit 31. When step ST1 is started, the biological sensor 11 continues to acquire the biological information until the power is turned off. In addition, the biological sensor 11 continues to transmit the acquired biological information to the processing unit 21.

In the first embodiment, the biological sensor 11 is a capacitive sensor. The biological sensor 11 acquires information on a capacitance as biological information. In addition, the biological sensor 11 transmits the information on the capacitance to the processing unit 21. The processing unit 21 receives the information on the capacitance from the biological sensor 11, and converts the capacitance into a frequency by the frequency conversion circuit. Therefore, the processing unit 21 outputs a value of the frequency as the output value of the biological sensor 11. In addition, the processing unit 21 continues to output the value of the frequency as the output value of the biological sensor 11 while receiving the information on the capacitance from the biological sensor 11.

In step ST2, the contact detection unit 12 acquires contact information between the measurement site and the contact surface 10a. The contact information acquired by the contact detection unit 12 is transmitted to the processing unit 21.

In the first embodiment, step ST2 is started by turning on the power at the operation display unit 31. When step ST2 is started, the contact detection unit 12 continues to acquire the contact information until the power is turned off. In addition, the contact detection unit 12 continues to transmit the acquired contact information to the processing unit 21.

In the first embodiment, the contact detection unit 12 includes the light sensor that detects photovoltaic power. The contact detection unit 12 acquires information on photovoltaic power as contact information. In addition, the contact detection unit 12 transmits the information on the photovoltaic power to the processing unit 21. The processing unit 21 receives the value of the photovoltaic power as the output value of the contact detection unit 12.

In step ST3, the processing unit 21 detects the contact between the measurement site and the contact surface 10a on the basis of the output value of the biological sensor 11 and the output value of the contact detection unit 12. Specifically, the processing unit 21 determines whether or not the measurement site and the contact surface 10a are in contact with each other on the basis of the first threshold value S1 of the output value of the contact detection unit 12 and the second threshold value S2 of the output value of the biological sensor 11.

In step ST3, “determines whether or not the measurement site and the contact surface 10a are in contact with each other” means determining whether or not the contact is to the extent that measurement accuracy can be guaranteed. In a case where the measurement site and a part of the contact surface 10a are in contact with each other, there may be a case where the processing unit 21 determines that the measurement site and the contact surface 10a are not in contact with each other. For example, in a case where the measurement site and a part of the contact surface 10a are in point contact with each other, the processing unit 21 may determine that they are not in contact with each other. On the other hand, in a case where the measurement site and the entire contact surface 10a are in surface contact with each other, the processing unit 21 may determine that they are in contact with each other.

When the processing unit 21 determines that the measurement site and the contact surface 10a are in contact with each other, that is, when “Yes” in step ST3, the processing proceeds to step ST4. When the processing unit 21 determines that the measurement site and the contact surface 10a are not in contact with each other, that is, when “No” in step ST3, the processing in step ST3 is repeated.

As illustrated in FIG. 7, step ST3 includes step ST3A and step ST3B.

In step ST3A, the processing unit 21 determines whether or not the output value of the contact detection unit 12 is equal to or less than the first threshold value S1. When the processing unit 21 determines that the output value of the contact detection unit 12, that is, the value of the photovoltaic power is equal to or less than the first threshold value S1, the processing proceeds to step ST3B. When the processing unit 21 determines that the output value of the contact detection unit 12, that is, the value of the photovoltaic power is greater than the first threshold value S1, the processing in step ST3A is repeated.

In step ST3B, the processing unit 21 determines whether or not the output value of the biological sensor 11 is equal to or less than the second threshold value S2. When the processing unit 21 determines that the output value of the biological sensor 11, that is, the value of the frequency is equal to or less than the second threshold value S2, the processing proceeds to step ST4. When the processing unit 21 determines that the output value of the biological sensor 11, that is, the value of the frequency is greater than the second threshold value S2, the processing returns to step ST3A.

In this manner, by performing steps ST3A and ST3B, the processing unit 21 detects the contact between the measurement site and the contact surface 10a. In addition, the processing unit 21 detects the contact between the measurement site and the contact surface 10a on the basis of the first threshold value S1 of the output value of the contact detection unit 12 and the second threshold value S2 of the output value of the biological sensor 11. As a result, the measurement accuracy can be improved.

Returning to FIG. 6, in step ST4, the processing unit 21 outputs trigger information for starting processing of calculating the amount of the measurement object. For example, the processing unit 21 outputs the trigger information to the calculation unit provided in the intraoral measurement device 1A. Alternatively, the processing unit 21 outputs the trigger information to the calculation unit provided in a device different from the intraoral measurement device 1A.

The calculation unit starts the processing of calculating the amount of the measurement object on the basis of the trigger information. In the first embodiment, the amount of the measurement object is a moisture amount. The calculation unit calculates the moisture amount on the basis of the output value of the biological sensor 11, that is, the value of the frequency.

The information on the amount of the measurement object calculated by the calculation unit is transmitted to the operation display unit 31. The operation display unit 31 displays the information on the amount of the measurement object.

In this manner, by performing steps ST1 to ST4, the intraoral measurement device 1A can detect the contact between the measurement site and the contact surface 10a, and can output the trigger information for starting the processing of calculating the amount of the measurement object.

### [Examples of Contact Detection Processing]

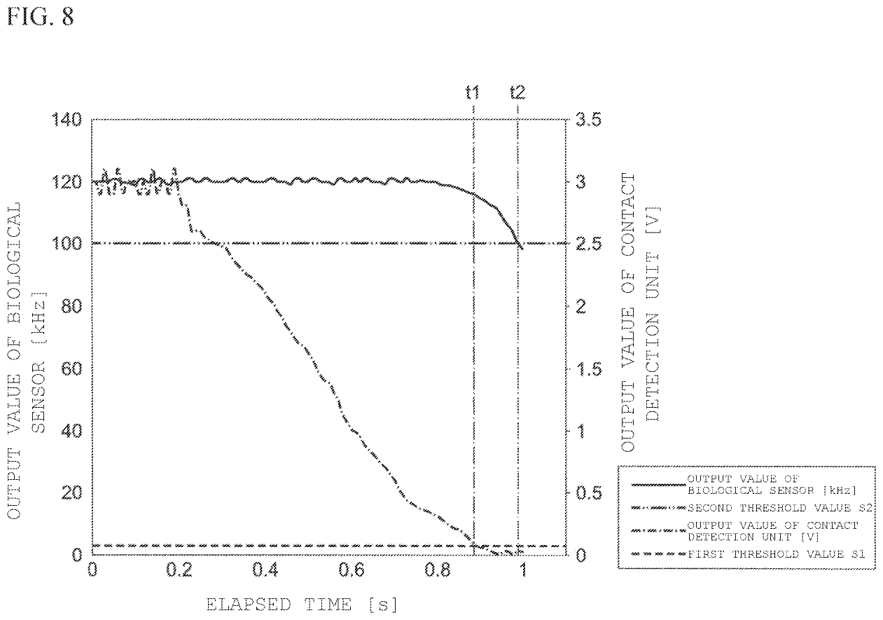

Examples of contact detection processing will be described. FIGS. 8 to 10 are schematic views illustrating examples of the contact detection processing. The contact detection processing illustrated in FIGS. 8 to 10 illustrates step ST3 in FIG. 6.

FIG. 8 illustrates an example of the contact detection processing in a case where the contact surface 10a of the intraoral measurement device 1A is brought into contact with a tongue portion of a user in a dry state.

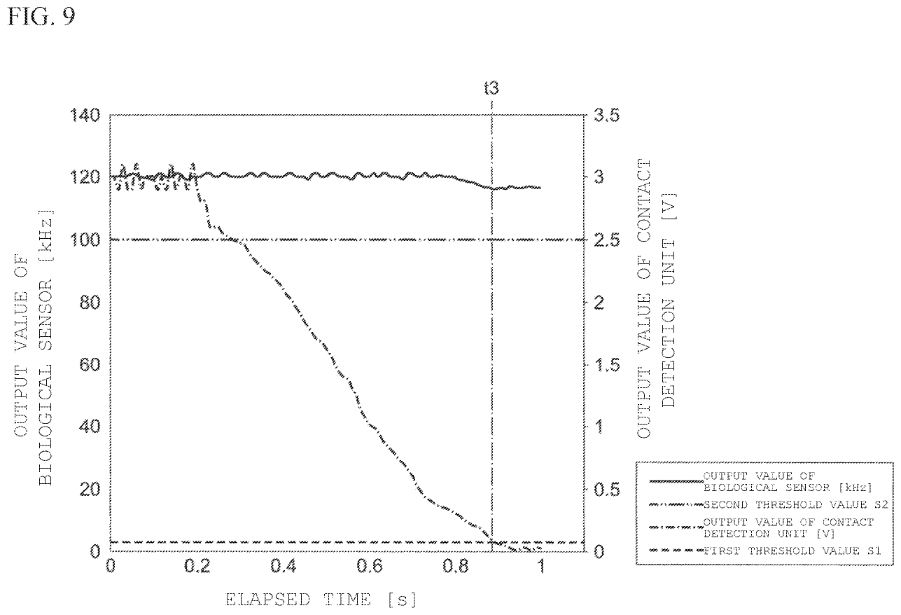

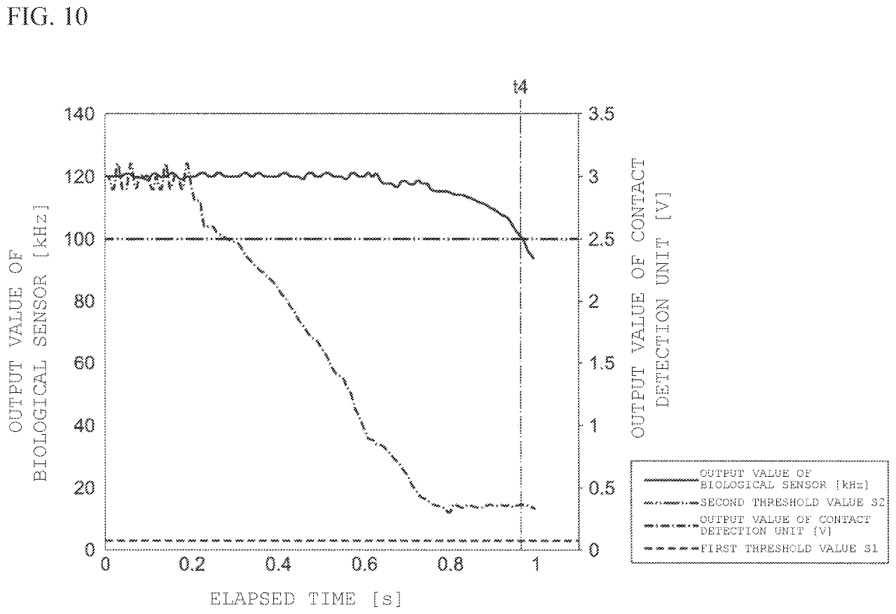

Note that the dry state means a state in which the tongue portion is dry. FIG. 9 illustrates an example of the contact detection processing in a case where the contact surface 10a of the intraoral measurement device 1A is brought into contact with an insulator. FIG. 10 illustrates an example of the contact detection processing in a case where a tongue portion of a user in a wet state and the contact surface 10a of the intraoral measurement device 1A are not in contact with each other. Note that the wet state means a state in which the tongue is wet.

In the examples illustrated in FIGS. 8 to 10, the first threshold value S1 is set to 0.1 V, and the second threshold value S2 is set to 100 kHz. Note that the first threshold value S1 and the second threshold value S2 are not limited to these values. The first threshold value S1 and the second threshold value S2 can be set to optional values.

In the examples illustrated in FIGS. 8 and 9, the contact surface 10a of the intraoral measurement device 1A is brought close to the contact target from a point 10 mm away from the contact target at a speed of 10 mm/s, and is brought into contact with the contact target. In the example illustrated in FIG. 10, the contact surface 10a of the intraoral measurement device 1A is brought close to the contact target from a point 10 mm away from the contact target at a speed of 10 mm/s, but is not brought into contact with the contact target. In the examples illustrated in FIGS. 8 to 10, the processing unit 21 continues to output the value of photovoltaic power as the output value of the contact detection unit 12. In addition, the processing unit 21 continues to output the value of the frequency as the output value of the biological sensor 11.

In the example illustrated in FIG. 8, the contact target is a tongue portion of a user in a dry state. As illustrated in FIG. 8, when the contact surface 10a of the intraoral measurement device 1A is gradually brought close to and brought into contact with the tongue portion of the user, the output value of the contact detection unit 12 gradually decreases. Specifically, as the contact surface 10a comes close to the tongue portion of the user, light to enter the contact detection unit 12 is gradually blocked by the tongue portion of the user. Therefore, the output value of the contact detection unit 12, that is, the value of the photovoltaic power decreases.

At a timing t1 illustrated in FIG. 8, substantially the entire contact detection unit 12 comes into contact with the tongue portion of the user. As a result, the output value of the contact detection unit 12 becomes equal to or less than the first threshold value S1. At this time, the output value of the biological sensor 11 is greater than the second threshold value S2. Therefore, the processing unit 21 does not detect the contact between the tongue portion of the user and the contact surface 10a.

At a timing t2 illustrated in FIG. 8, the output value of the contact detection unit 12 is equal to or less than the first threshold value S1, and the output value of the biological sensor 11 is equal to or less than the second threshold value S2. At this time, the processing unit 21 detects the contact between the tongue portion of the user and the contact surface 10a.

In this manner, the processing unit 21 detects the contact between the tongue portion of the user and the contact surface 10a when the output value of the contact detection unit 12 becomes equal to or less than the first threshold value S1, and the output value of the biological sensor 11 becomes equal to or less than the second threshold value S2. When detecting the contact, the processing unit 21 outputs the trigger information.

In the example illustrated in FIG. 9, the contact target is an insulator such as a desk. As illustrated in FIG. 9, when the contact surface 10a of the intraoral measurement device 1A is gradually brought close to and brought into contact with the insulator, the output value of the contact detection unit 12 decreases as in the example illustrated in FIG. 8.

After a timing t3 illustrated in FIG. 9, the output value of the contact detection unit 12 is equal to or less than the first threshold value S1, while the output value of the biological sensor 11 is greater than the second threshold value S2. Therefore, the processing unit 21 does not detect the contact between the insulator and the contact surface 10a. As a result, the processing unit 21 does not output the trigger information in a case where the contact target is not the measurement site in an oral cavity.

In the example illustrated in FIG. 10, the contact target is a tongue portion of a user in a wet state, but the contact surface 10a of the intraoral measurement device 1A and the tongue portion of the user are not brought into contact with each other. As illustrated in FIG. 10, when the contact surface 10a of the intraoral measurement device 1A is gradually brought close to the tongue portion of the user, the output value of the contact detection unit 12 gradually decreases. However, the output value of the contact detection unit 12 is greater than the first threshold value S1.

On the other hand, as the contact surface 10a of the intraoral measurement device 1A comes close to the tongue portion of the user, the output value of the biological sensor 11 decreases. At a timing t4 illustrated in FIG. 10, the output value of the biological sensor 11 becomes equal to or less than the second threshold value S2.

The processing unit 21 does not detect the contact between the tongue portion of the user and the contact surface 10a because the output value of the contact detection unit 12 is greater than the first threshold value S1. As a result, the processing unit 21 does not output the trigger information in a case where the contact surface 10a and the tongue portion of the user are not in contact with each other, or in a case where the contact is insufficient. [Method for using intraoral measurement device]

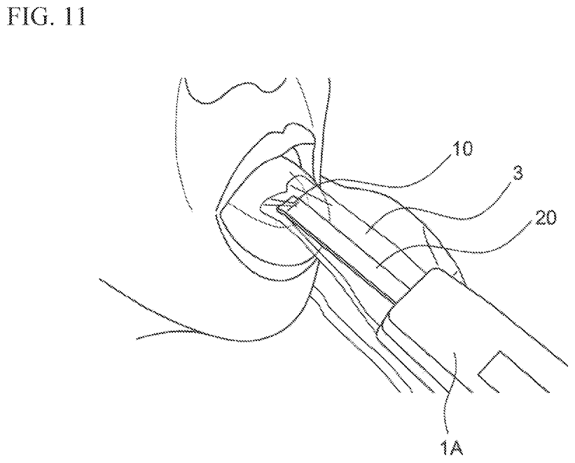

An example of a method for using the intraoral measurement device 1A will be described with reference to FIG. 11. FIG. 11 is a schematic view illustrating an example of a mode in which the intraoral measurement device 1A of the first embodiment according to the present disclosure is used.

As illustrated in FIG. 11, the sensor unit 10 and the probe unit 20 of the intraoral measurement device 1A are covered with a film 3. Pressing the power button of the operation display unit 31 turns on the power of the intraoral measurement device 1A. This brings the intraoral measurement device 1A into a measurable state.

In the measurement, the contact surface 10a of the intraoral measurement device 1A is brought into contact with a measurement site in an oral cavity of a user. For example, the contact surface 10a is brought into contact with a tongue portion of the user.

In the intraoral measurement device 1A, the example of the operation illustrated in FIG. 6 is performed. That is, in the intraoral measurement device 1A, the processing unit 21 outputs trigger information for starting processing of calculating an amount of a measurement object on the basis of an output value of the biological sensor 11 and an output value of the contact detection unit 12. The trigger information is transmitted to the calculation unit. The calculation unit performs the processing of calculating the amount of the measurement object on the basis of the trigger information.

When the measurement ends, information on the amount of the measurement object is displayed on the operation display unit 31 as a measurement result. For example, the intraoral measurement device 1A may be provided with a speaker, and a user may be notified of the end of the measurement by voice information from the speaker.

The intraoral measurement device 1A according to the first embodiment can acquire the following effects.

The intraoral measurement device 1A has the contact surface 10a that comes into contact with a measurement site in an oral cavity. The intraoral measurement device 1A includes the biological sensor 11, the contact detection unit 12, and the processing unit 21. The biological sensor 11 is arranged on the contact surface 10a, and has the detection surface 11a that acquires biological information. The contact detection unit 12 acquires contact information that indicates the degree of contact between the measurement site and the contact surface 10a. The processing unit 21 outputs trigger information for starting processing of calculating the amount of the measurement object on the basis of the output value of the biological sensor 11 and the output value of the contact detection unit 12.

In this manner, the intraoral measurement device 1A outputs the trigger information on the basis of the output value of the biological sensor 11 and the output value of the contact detection unit 12. Therefore, the contact to the extent that the measurement accuracy can be guaranteed can be detected, so that the measurement accuracy of the intraoral measurement device 1A can be improved.

Further, the contact between the measurement site in the oral cavity and the contact surface 10a can be easily detected. By detecting the contact between the measurement site in the oral cavity and the contact surface 10a, the contact between the measurement site and the detection surface 11a of the biological sensor 11 can be easily detected.

The processing unit 21 outputs the trigger information when the output value of the contact detection unit 12 is equal to or less than the first threshold value S1, and the output value of the biological sensor 11 is equal to or less than the second threshold value S2. With such a configuration, the contact between the measurement site in the oral cavity and the contact surface 10a can be easily detected with high accuracy. As a result, the measurement accuracy of the intraoral measurement device 1A can be further improved.

Note that the output of the trigger information from the processing unit 21 may be performed on the basis of the variation range of the output value of the biological sensor 11 and the variation range of the output value of the contact detection unit 12. That is, the processing unit 21 may detect the contact between the measurement site in the oral cavity and the contact surface 10a on the basis of the variation range of the output value of the biological sensor 11 and the variation range of the output value of the contact detection unit 12. With such a configuration, the contact can be detected without necessarily being affected by the individual difference between users. As a result, the measurement accuracy of the intraoral measurement device 1A can be further improved.

The intraoral measurement device 1A includes the housing 2 that has a rod shape and houses the biological sensor 11 and the contact detection unit 12. The contact surface 10a is provided at the one end E1 side in the longitudinal direction D1 of the housing 2, and is provided in a direction (X, Y direction) intersecting the end surface on the one end E1 side. With such a configuration, the measurement site in the oral cavity and the contact surface 10a can be more easily brought into contact with each other.

The contact detection unit 12 includes the optical sensor 13 that receives light and the light guide unit 14 that is provided on the contact surface 10a and guides light to the optical sensor. With such a configuration, the measurement accuracy of the intraoral measurement device 1A can be further improved with a simple configuration.

The optical sensor 13 includes the light emitting unit 15 that emits light and the light receiving unit 16 that receives light reflected by the measurement site. With such a configuration, the measurement accuracy of the intraoral measurement device 1A can be further improved.

The biological sensor 11 is a capacitive sensor that detects a capacitance. The output value of the biological sensor 11 is a frequency obtained by processing of converting the capacitance detected by the capacitive sensor. With such a configuration, the measurement accuracy of the intraoral measurement device 1A can be further improved.

The intraoral measurement device 1A is provided with the operation display unit 31 that displays information on the amount of the measurement object. With such a configuration, the information on the amount of the measurement object, which is a measurement result, can be easily checked.

The contact detection unit 12 is arranged at the periphery of the biological sensor 11. With such a configuration, the measurement accuracy of the intraoral measurement device 1A can be further improved.

The contact detection unit 12 is arranged at a position farther from the one end E1 in the longitudinal direction D1 of the housing 2 than the biological sensor 11. With such a configuration, the separation of the contact surface 10a with respect to the measurement site can be easily detected, so that the contact between the measurement site and the contact surface 10a can be detected with high accuracy. As a result, the measurement accuracy of the intraoral measurement device 1A can be improved.

Note that, in the first embodiment, an example has been described in which the intraoral measurement device 1A includes the biological sensor 11, the contact detection unit 12, the processing unit 21, and the operation display unit 31, but the present disclosure is not limited thereto. In the intraoral measurement device 1A, these constituent elements may be implemented by one device, or be implemented by a plurality of devices. For example, the processing unit 21 and the operation display unit 31 may be integrally formed. The biological sensor 11 and the processing unit 21 may be integrally formed.

In the first embodiment, an example has been described in which the operation display unit 31 is provided in the intraoral measurement device 1A, but the present disclosure is not limited thereto. The operation display unit 31 may not be provided in the intraoral measurement device 1A. For example, the operation display unit 31 may be provided in a processing device different from the intraoral measurement device 1A.

In the first embodiment, an example has been described in which the measurement object of the intraoral measurement device 1A is moisture, and the intraoral measurement device 1A measures the moisture amount in an oral cavity, but the present disclosure is not limited thereto. It is sufficient that the intraoral measurement device 1A can measure the state in an oral cavity. For example, the intraoral measurement device 1A may measure a secretion amount of saliva, bite force, tongue pressure, tongue color and/or amounts of various substances contained in saliva. Specifically, the intraoral measurement device 1A may measure secretion amounts of electrolytes, various enzymes, proteins, ammonia, and the like, as measurement objects.

In the first embodiment, an example has been described in which the housing 2 includes the sensor unit 10, the probe unit 20, and the grip unit 30, but the present disclosure is not limited thereto. It is sufficient that the housing 2 extends in the longitudinal direction.

In the first embodiment, an example has been described in which the biological sensor 11 is a capacitive sensor, but the present disclosure is not limited thereto. It is sufficient that the biological sensor 11 is a sensor that can acquire biological information. For example, the biological sensor 11 may be at least one of an impedance measurement sensor, a load sensor, and a humidity sensor.

In the first embodiment, an example has been described in which the detection surface 11a of the biological sensor 11 is formed in a rectangular shape as viewed in the height direction (Z direction) of the intraoral measurement device 1A, but the present disclosure is not limited thereto. For example, the detection surface 11a of the biological sensor may have a polygonal shape, circular shape, or an elliptical shape as viewed in the height direction (Z direction) of the intraoral measurement device TA.

In the first embodiment, an example has been described in which the contact detection unit 12 includes the optical sensor 13 and the light guide unit 14, but the present disclosure is not limited thereto. It is sufficient that the contact detection unit 12 includes a sensor that acquires contact information between the measurement site and the contact surface 10a. For example, the contact detection unit 12 may include a contact sensor or an acoustic sensor. As the contact sensor, for example, a conductive sensor, a capacitive sensor, a resistive film type contact sensor, and a thermistor type temperature sensor can be named. As the acoustic sensor, for example, an ultrasonic type distance sensor can be named. The contact sensor and the acoustic sensor are arranged so as to be exposed to the contact surface 10a. The contact detection unit 12 may not include the light guide unit 14.

In the first embodiment, an example has been described in which the optical sensor 13 is a light sensor, but the present disclosure is not limited thereto. For example, the optical sensor 13 may be an infrared sensor or a laser distance sensor.

In the first embodiment, an example has been described in which the intraoral measurement device 1A includes one contact detection unit 12, but the present disclosure is not limited thereto. The intraoral measurement device 1A may include one or a plurality of contact detection units 12. In a case where the intraoral measurement device 1A includes a plurality of contact detection units 12, the plurality of contact detection units 12 may be configured by combining an optical sensor, a contact sensor, and an acoustic sensor. By combining a plurality of types of sensors, the measurement accuracy can be further improved. Alternatively, a plurality of contact detection units 12 may be configured by one type of sensor.

In the first embodiment, an example has been described in which the contact detection unit 12 is arranged on the center C1 side of the housing 2 with respect to the biological sensor 11 in the longitudinal direction D1 of the intraoral measurement device 1A, but the present disclosure is not limited thereto. Further, an example has been described in which the contact detection unit 12 is arranged at the center in the width direction (X direction) of the intraoral measurement device 1A as viewed in the height direction (Z direction) of the intraoral measurement device 1A, but the present disclosure is not limited thereto. It is sufficient that the contact detection unit 12 is arranged at a position where contact between the measurement site and the contact surface 10a can be detected.

In the first embodiment, an example has been described in which the contact detection unit 12 is arranged at the periphery of the biological sensor 11, but the present disclosure is not limited thereto. The contact detection unit 12 may be arranged on the biological sensor 11. For example, in a case where the biological sensor 11 has translucency, the contact detection unit 12 may be arranged on the biological sensor 11. In this case, the contact detection unit 12 may use the biological sensor 11 as the light guide unit 14, and be arranged on an arrangement surface on a side opposite to the detection surface 11a of the biological sensor 11.

In the first embodiment, an example has been described in which the processing unit 21 includes the conversion circuit that performs processing of converting a capacitance into a frequency, but the present disclosure is not limited thereto. The processing unit 21 may include a circuit that converts biological information acquired by the biological sensor 11 into information other than a frequency. Alternatively, the processing unit 21 may not include the conversion circuit. In this case, biological information acquired by the biological sensor 11 is directly output as the output value of the biological sensor 11. That is, the output value of the biological sensor 11 is a value of a capacitance.

In the first embodiment, an example has been described in which the operation display unit 31 includes the operation unit and the display unit, but the present disclosure is not limited thereto. It is sufficient that the operation display unit 31 includes at least one of the operation unit and the display unit.

In the first embodiment, steps ST1 to ST4 illustrated in FIG. 6 have been used to describe the example of the operation of the intraoral measurement device 1A, but the present disclosure is not limited thereto. For example, steps ST1 to ST4 illustrated in FIG. 6 may be integrated or divided. Alternatively, the flowchart illustrated in FIG. 6 may include additional steps. For example, a step of displaying a measurement result on the operation display unit 31 may be added.

In the first embodiment, steps ST3A and ST3B illustrated in FIG. 7 have been used to describe the example of the contact detection processing, but the present disclosure is not limited thereto. For example, steps ST3A and ST3B illustrated in FIG. 7 may be integrated or divided. Alternatively, the order of steps ST3A and ST3B may be switched. The flowchart illustrated in FIG. 7 may include additional steps.

### (Second Embodiment)

An intraoral measurement device according to a second embodiment of the present disclosure will be described. Note that, in the second embodiment, points different from the first embodiment will be mainly described. In the second embodiment, the same or equivalent configurations as those in the first embodiment will be described with the same reference numerals. In addition, in the second embodiment, descriptions overlapping with those in the first embodiment are omitted.

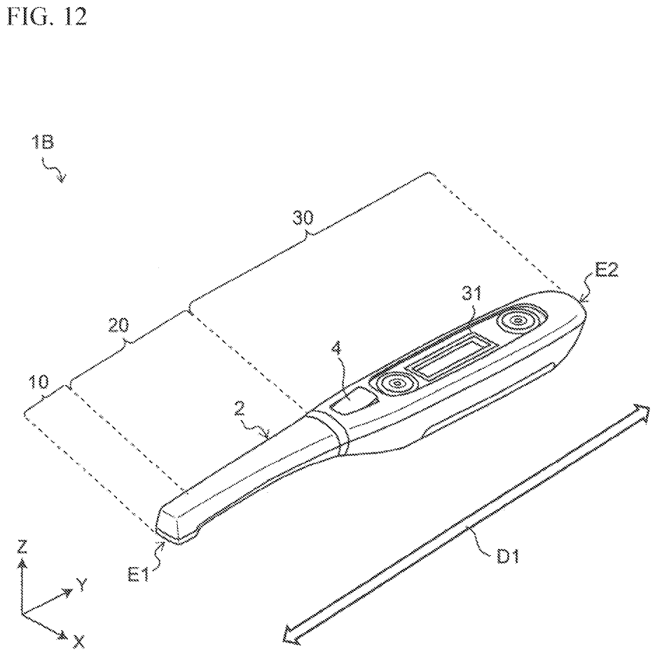

An example of the intraoral measurement device according to the second embodiment will be described with reference to FIG. 12. FIG. 12 is a schematic perspective view of an example of an intraoral measurement device 1B of the second embodiment according to the present disclosure.

The second embodiment is different from the first embodiment in that a mechanical switch 4 is provided.

As illustrated in FIG. 12, the intraoral measurement device 1B includes the mechanical switch 4. The mechanical switch 4 is actuated by pressing the contact surface 10a of the intraoral measurement device 1B. Specifically, pressing the contact surface 10a turns on the mechanical switch 4.

In this specification, “pressing” means applying force to a target irrespective of the degree of contact. For example, “pressing” is a state in which force is applied to a target irrespective of point contact or surface contact. In addition, in this specification, “contact” means coming into contact with the target irrespective of the force. For example, “contact” is a state of being in surface contact with a target instead of point contact.

The mechanical switch 4 includes a swing unit that swings the sensor unit 10 and the probe unit 20 with respect to the grip unit 30. The swing unit supports the sensor unit 10 and the probe unit 20, and swings the sensor unit 10 and the probe unit 20 about a predetermined swing center with respect to the grip unit 30.

When the contact surface 10a is pressed against the contact target, the swing unit swings the sensor unit 10 and the probe unit 20 about the swing center with respect to the grip unit 30. The swing unit swings the sensor unit 10 and the probe unit 20 in a range of 5° or more and 15° or less in the height direction (Z direction) of the intraoral measurement device 1B. When the swing unit swings the sensor unit 10 and the probe unit 20, the mechanical switch 4 is turned on.

When the contact surface 10a is not pressed against the contact target, the swing unit does not swing, so that the mechanical switch 4 is off.

The processing unit 21 performs contact detection processing on the basis of the operation state of the mechanical switch 4. That is, the processing unit 21 outputs trigger information on the basis of the operation state of the mechanical switch 4 in addition to the output value of the biological sensor 11 and the output value of the contact detection unit 12.

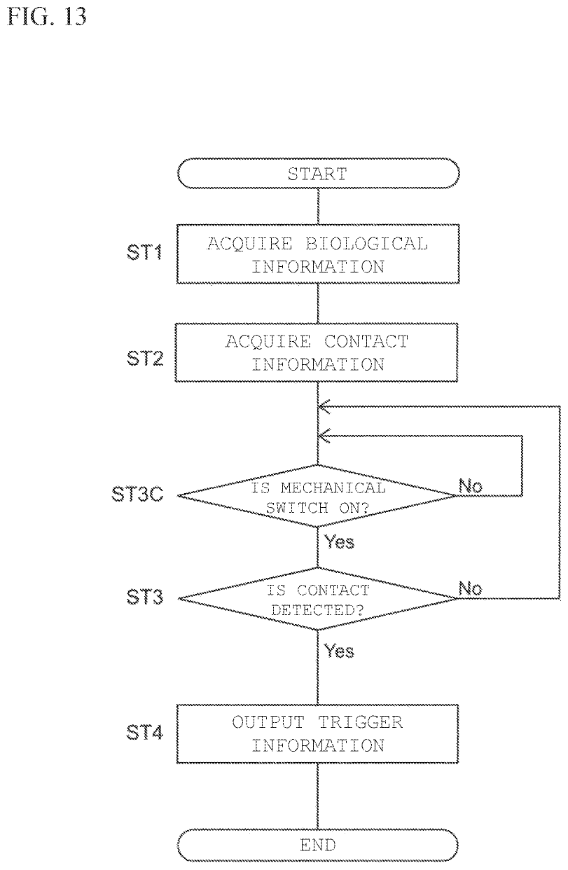

FIG. 13 is a flowchart illustrating an example of an operation of the intraoral measurement device 1B of the second embodiment according to the present disclosure. Steps ST1 to ST4 illustrated in FIG. 13 are substantially the same as steps ST1 to ST4 illustrated in FIG. 6 in the first embodiment, and thus description thereof is omitted.

As illustrated in FIG. 13, the operation of the intraoral measurement device 1B includes step ST3C of determining whether or not the mechanical switch 4 is on, in addition to steps ST1 to ST4. Step ST3C is performed before step ST3 in which the contact detection processing is performed.

In step ST3C, the processing unit 21 determines whether or not the mechanical switch 4 is on. When the processing unit 21 determines that the mechanical switch 4 is on, the processing proceeds to step ST3. When the processing unit 21 determines that the mechanical switch 4 is off, the processing in step ST3C is repeated.

In this manner, by performing step ST3C in addition to steps ST1 to ST4, the intraoral measurement device 1B can easily detect the contact between the measurement site and the contact surface 10a with higher accuracy, and can output the trigger information for starting the processing of calculating the amount of the measurement object.

The intraoral measurement device 1B according to the second embodiment can acquire the following effects.

The intraoral measurement device 1B includes the mechanical switch 4 that is actuated by pressing the contact surface 10a. The processing unit 21 outputs trigger information on the basis of the operation state of the mechanical switch 4. With such a configuration, the contact between the measurement site and the contact surface 10a can be easily detected with higher accuracy. As a result, the measurement accuracy can be improved. In addition, by performing the contact detection processing on the basis of the operation state of the mechanical switch 4, the measurement can be performed at a timing desired by a user. As a result, usability for the user can be improved.

Note that, in the second embodiment, an example has been described in which the mechanical switch 4 includes the swing unit, but the present disclosure is not limited thereto. It is sufficient that the mechanical switch 4 has a mechanism that is actuated by pressing the contact surface 10a.

In the second embodiment, an example has been described in which step ST3C is performed before step ST3, but the present disclosure is not limited thereto. For example, step ST3C may be integrated with step ST3. Step ST3C may be performed between steps ST3A and ST3B.

### (Third Embodiment)

An intraoral measurement device according to a third embodiment of the present disclosure will be described. Note that, in the third embodiment, points different from the first embodiment will be mainly described. In the third embodiment, the same or equivalent configurations as those in the first embodiment will be described with the same reference numerals. In addition, in the third embodiment, descriptions overlapping with those in the first embodiment are omitted.

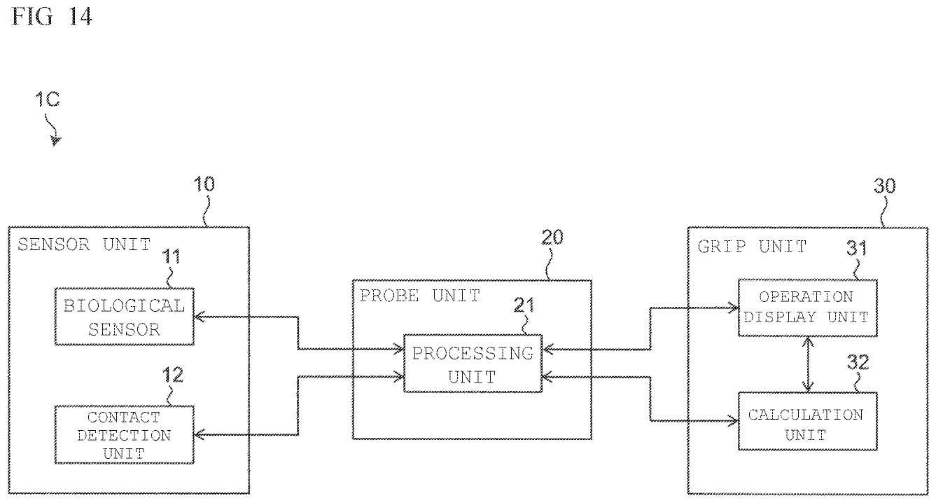

An example of the intraoral measurement device according to the third embodiment will be described with reference to FIG. 14. FIG. 14 is a block diagram illustrating a schematic configuration of an example of an intraoral measurement device 1C of the third embodiment according to the present disclosure.

The third embodiment is different from the first embodiment in that a calculation unit 32 is provided.

As illustrated in FIG. 14, the intraoral measurement device 1C is provided with the calculation unit 32. The calculation unit 32 calculates the amount of the measurement object on the basis of the output value of the biological sensor 11. In addition, the calculation unit 32 starts calculation of the amount of the measurement object on the basis of the trigger information.

The calculation unit 32 is housed in the grip unit 30 of the housing 2. The calculation unit 32 receives information on the output value of the biological sensor 11 and the trigger information from the processing unit 21. While the calculation unit 32 continues to receive information on the output value of the biological sensor 11 from the processing unit 21, the calculation unit 32 does not start calculation of the amount of the measurement object unless receiving the trigger information.

In the third embodiment, the calculation unit 32 starts the calculation of a moisture amount on the basis of the trigger information output from the processing unit 21. The calculation unit 32 calculates the moisture amount on the basis of the output value of the biological sensor 11, that is, information on a frequency.

The calculation unit 32 can be implemented by a semiconductor element or the like. The function of the calculation unit 32 may be configured only by hardware, or be implemented by combining hardware and software. The calculation unit 32 includes, for example, a moisture amount calculation circuit that calculates the moisture amount on the basis of the amount of change in frequency. Note that the amount of change in frequency is a difference between a reference frequency and a frequency converted by the processing unit 21 on the basis of information on a capacitance. The reference frequency means the frequency in a standard air atmosphere.

The calculation unit 32 includes a storage unit. The storage unit can be implemented by, for example, a hard disk (HDD), an SSD, a RAM, a DRAM, a ferroelectric memory, a flash memory, a magnetic disk, or combination thereof. For example, when performing calculation of the amount of the measurement object, the calculation unit 32 stores, in the storage unit, information on the output value of the biological sensor 11 transmitted from the processing unit 21.

The information on the moisture amount calculated by the calculation unit 32 is transmitted to the operation display unit 31.

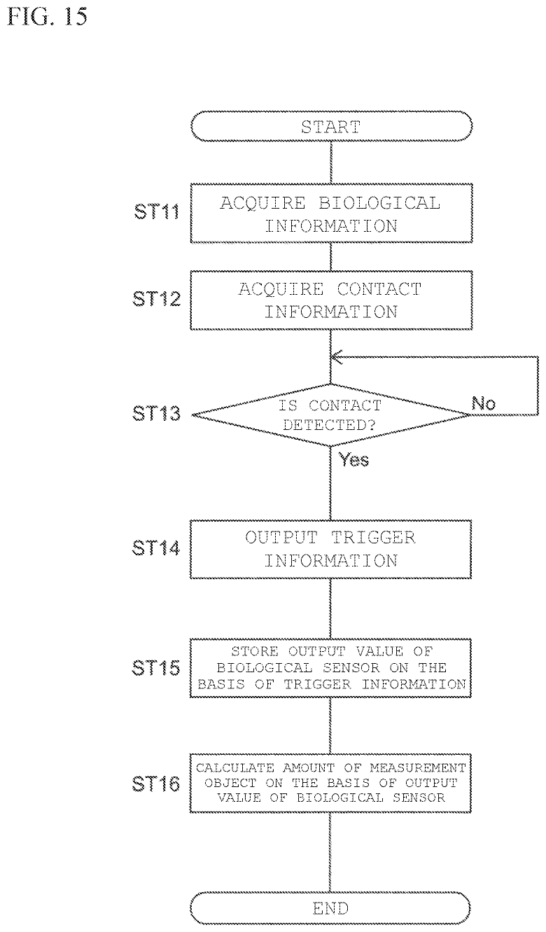

FIG. 15 is a flowchart illustrating an example of an operation of the intraoral measurement device 1C of the third embodiment according to the present disclosure. Steps ST11 to ST14 illustrated in FIG. 15 are substantially the same as steps ST1 to ST4 illustrated in FIG. 6 in the first embodiment, and thus description thereof is omitted.

As illustrated in FIG. 15, in step ST14, the processing unit 21 outputs the trigger information to the calculation unit 32. That is, the processing unit 21 transmits the trigger information to the calculation unit 32.

In step ST15, the calculation unit 32 stores, in the storage unit, the information on the output value of the biological sensor 11 on the basis of the trigger information. Specifically, when receiving the trigger information from the processing unit 21, the calculation unit 32 stores, in the storage unit, the information on the output value of the biological sensor 11 transmitted from the processing unit 21. For example, the calculation unit 32 stores, in the storage unit, the information on the output value of the biological sensor 11 for 1.5 seconds from a point of time at which the calculation unit 32 receives the trigger information. Alternatively, the output value of the biological sensor 11 may be stored as needed in a memory that can temporarily store data. Then, the information on the output value of the biological sensor 11 may be read from the memory for 1.0 seconds from 0.5 seconds before the point of time when receiving the trigger information, and be stored in the storage unit.

In step ST16, the calculation unit 32 calculates the amount of the measurement object on the basis of the output value of the biological sensor 11. Specifically, the calculation unit 32 calculates the amount of the measurement object on the basis of the output value of the biological sensor 11 stored in the storage unit.

In the third embodiment, the calculation unit 32 calculates the moisture amount on the basis of the information on the frequency stored in the storage unit.

The calculation unit 32 transmits the information on the calculated amount of the measurement object to the operation display unit 31. The operation display unit 31 receives and displays the information on the amount of the measurement object.

In this manner, by performing steps ST11 to ST16, the intraoral measurement device 1C can calculate the amount of the measurement object.

The intraoral measurement device 1C according to the third embodiment can acquire the following effects.

The intraoral measurement device 1C is provided with the calculation unit 32 that calculates the amount of the measurement object on the basis of the output value of the biological sensor 11. The calculation unit 32 starts calculation of the amount of the measurement object on the basis of the trigger information. With such a configuration, the contact between the measurement site and the contact surface 10a can be easily detected with high accuracy, and the amount of the measurement object can be calculated on the basis of the trigger information for starting processing of calculating the amount of the measurement object.

Note that, in the third embodiment, an example has been described in which the calculation unit 32 is arranged inside the grip unit 30, but the present disclosure is not limited thereto. For example, the calculation unit 32 may be arranged inside the probe unit 20. In this case, the calculation unit 32 may be integrally formed with the processing unit 21. For example, the processing unit 21 may include the frequency conversion circuit that converts information on a capacitance into a frequency and the moisture amount calculation circuit that calculates the moisture amount on the basis of the amount of change in frequency.

In the third embodiment, an example has been described in which the calculation unit 32 calculates the moisture amount as the amount of the measurement object, but the present disclosure is not limited thereto. In addition, an example has been described in which the calculation unit 32 includes the moisture amount calculation circuit that calculates the moisture amount on the basis of the amount of change in frequency, but the present disclosure is not limited thereto. For example, it is sufficient that the calculation unit 32 includes the calculation circuit that calculates the amount of the measurement object.

### (Fourth Embodiment)

An intraoral measurement system according to a fourth embodiment of the present disclosure will be described. Note that, in the fourth embodiment, points different from the first embodiment will be mainly described. In the fourth embodiment, the same or equivalent configurations as those in the first embodiment will be described with the same reference numerals. In addition, in the fourth embodiment, descriptions overlapping with those in the first embodiment are omitted.

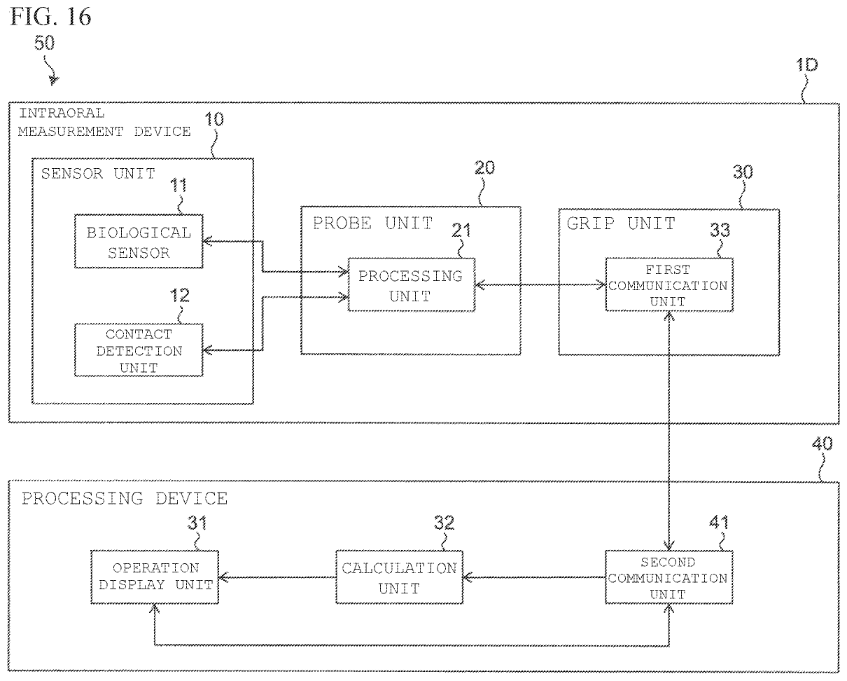

An example of the intraoral measurement system according to the fourth embodiment will be described with reference to FIG. 16. FIG. 16 is a block diagram illustrating a schematic configuration of an example of an intraoral measurement system 50 of the fourth embodiment according to the present disclosure.

The fourth embodiment is different from the first embodiment in that information acquired by an intraoral measurement device 1D is transmitted to a processing device 40, and the amount of the measurement object is calculated in the processing device 40.

As illustrated in FIG. 16, the intraoral measurement system 50 includes the intraoral measurement device 1D and the processing device 40. <Intraoral measurement device>

The intraoral measurement device 1D includes the biological sensor 11, the contact detection unit 12, the processing unit 21, and a first communication unit 33. In the fourth embodiment, the biological sensor 11, the contact detection unit 12, and the processing unit 21 are substantially the same as those in the first embodiment, and thus detailed description thereof is omitted.

The first communication unit 33 communicates with the processing device 40. Specifically, the first communication unit 33 transmits information on the output value of the biological sensor 11 and trigger information output from the processing unit 21 to the processing device 40.

The first communication unit 33 includes a circuit that performs communication with the processing device 40 in conformity with a predetermined communication standard. The predetermined communication standard includes, for example, a LAN, Wi-Fi (registered trademark), Bluetooth (registered trademark), a USB, an HDMI (registered trademark), a controller area network (CAN), a serial peripheral interface (SPI), a universal asynchronous receiver/transmitter (UART), and an inter-integrated circuit (I2C).

In the fourth embodiment, the processing unit 21 performs processing of converting a capacitance acquired by the biological sensor 11 into a frequency. The processing unit 21 transmits information on the frequency converted as the output value of the biological sensor 11 to the processing device 40 via the first communication unit 33.

The intraoral measurement device 1D includes a first control unit that comprehensively controls constituent elements constituting the intraoral measurement device 1D. The first control unit includes, for example, a memory that stores a program and a processing circuit that corresponds to a processor such as a central processing unit (CPU). For example, in the first control unit, the processor executes the program stored in the memory. In the fourth embodiment, the first control unit controls the biological sensor 11, the contact detection unit 12, the processing unit 21, and the first communication unit 33.

### <Processing Device>

The processing device 40 receives information from the intraoral measurement device 1D, and calculates an amount of a measurement object on the basis of the received information. Specifically, the processing device 40 starts calculation processing by receiving the trigger information from the intraoral measurement device 1D. In addition, the processing device 40 calculates the amount of the measurement object on the basis of the information on the output value of the biological sensor 11 received from the intraoral measurement device 1D. In the fourth embodiment, the processing device 40 calculates the moisture amount based on the information on the frequency received from the intraoral measurement device 1D.

The processing device 40 is a computer. For example, the processing device 40 may be a portable terminal such as a smartphone or a tablet terminal. Alternatively, the processing device 40 may be a server connected to a network.

The processing device 40 includes a second communication unit 41, the operation display unit 31, and the calculation unit 32. In the fourth embodiment, the operation display unit 31 and the calculation unit 32 are substantially the same as those in the embodiments 1 and 3, and thus detailed description thereof is omitted.

The second communication unit 41 communicates with the intraoral measurement device 1D. Specifically, the second communication unit 41 receives the information on the output value of the biological sensor 11 and the trigger information from the first communication unit 33 of the intraoral measurement device 1D.

The second communication unit 41 includes a circuit that performs communication with the intraoral measurement device 1D in conformity with a predetermined communication standard. The predetermined communication standard includes, for example, a LAN, Wi-Fi (registered trademark), Bluetooth (registered trademark), a USB, an HDMI (registered trademark), a controller area network (CAN), a serial peripheral interface (SPI), a universal asynchronous receiver/transmitter (UART), and an inter-integrated circuit (I2C).

The processing device 40 receives the information on the output value of the biological sensor 11 and the trigger information from the intraoral measurement device 1D via the second communication unit 41. In the fourth embodiment, the processing device 40 receives the information on the frequency and the trigger information from the intraoral measurement device 1D via the second communication unit 41.

In the processing device 40, the calculation unit 32 starts calculation of the amount of the measurement object on the basis of the trigger information received from the intraoral measurement device 1D. The calculation unit 32 calculates the amount of the measurement object on the basis of the information on the output value of the biological sensor 11 received from the intraoral measurement device 1D. In the fourth embodiment, the calculation unit 32 calculates the moisture amount on the basis of the information on the frequency. The information on the calculated moisture amount is transmitted to the operation display unit 31. The operation display unit 31 displays the information on the calculated moisture amount.

The processing device 40 includes a second control unit that comprehensively controls constituent elements constituting the processing device 40. The second control unit includes, for example, a memory that stores a program and a processing circuit that corresponds to a processor such as a central processing unit (CPU). For example, in the second control unit, the processor executes the program stored in the memory. In the fourth embodiment, the second control unit controls the second communication unit 41, the operation display unit 31, and the calculation unit 32.

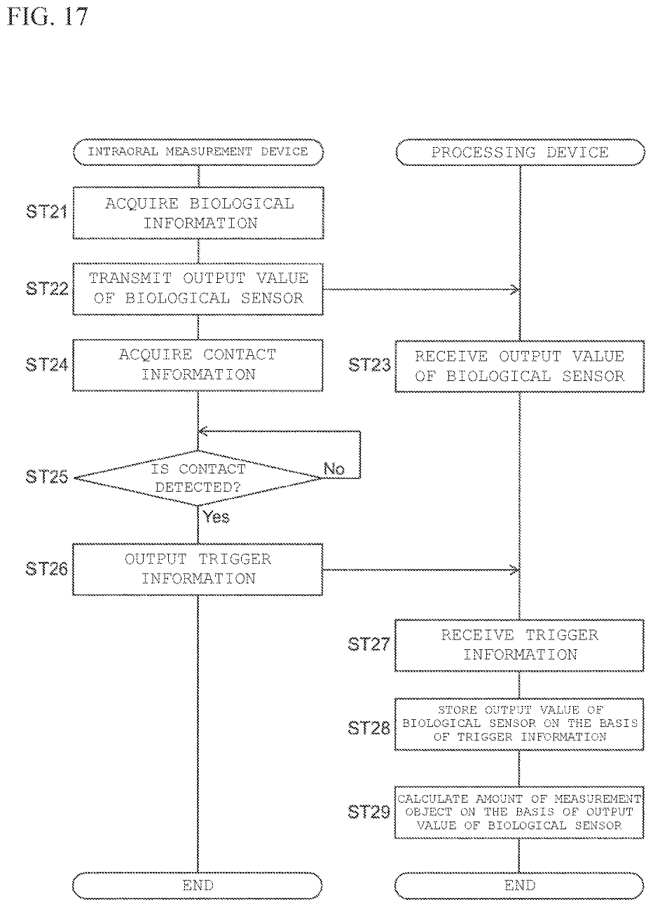

FIG. 17 is a flowchart illustrating an example of an operation of the intraoral measurement system 50 of the fourth embodiment according to the present disclosure. Steps ST21 and ST24 to ST26 illustrated in FIG. 17 are substantially the same as steps ST1 to ST4 illustrated in FIG. 6 in the first embodiment, and thus detailed description thereof is omitted.

As illustrated in FIG. 17, in step ST21, the biological sensor 11 acquires biological information. The biological sensor 11 acquires a capacitance as biological information. The biological sensor 11 transmits the information on the capacitance to the processing unit 21. The processing unit 21 converts the capacitance into a frequency.

In step ST22, the first communication unit 33 transmits the output value of the biological sensor 11 to the processing device 40. Specifically, the first communication unit 33 transmits the information on the frequency converted by the processing unit 21 to the processing device 40.

In step ST23, the second communication unit 41 of the processing device 40 receives the information on the output value of the biological sensor 11. Specifically, the second communication unit 41 receives the information on the frequency transmitted from the first communication unit 33 of the intraoral measurement device 1D.

In step ST24, the contact detection unit 12 acquires contact information that indicates the degree of contact between the measurement site and the contact surface 10a. In the fourth embodiment, the contact detection unit 12 acquires photovoltaic power.

In step ST25, the processing unit 21 detects the contact between the measurement site and the contact surface 10a. Specifically, the processing unit 21 detects the contact between the measurement site and the contact surface 10a on the basis of the output value of the biological sensor 11 and the output value of the contact detection unit 12. For example, the processing unit 21 determines whether or not the measurement site and the contact surface 10a are in contact with each other on the basis of the first threshold value S1 of the output value of the contact detection unit 12 and the second threshold value S2 of the output value of the biological sensor 11.

In step ST25, when the processing unit 21 determines that the measurement site and the contact surface 10a are in contact with each other, that is, when “Yes” in step ST25, the processing proceeds to step ST26. When the processing unit 21 determines that the measurement site and the contact surface 10a are not in contact with each other, that is, when “No” in step ST25, the processing in step ST25 is repeated.

In step ST26, the first communication unit 33 outputs trigger information to the processing device 40. Specifically, the processing unit 21 transmits the trigger information to the processing device 40 via the first communication unit 33.

In step ST27, the second communication unit 41 of the processing device 40 receives the trigger information. Specifically, the processing device 40 receives the trigger information from the intraoral measurement device 1D via the second communication unit 41.

In step ST28, the calculation unit 32 stores, in the storage unit, the information on the output value of the biological sensor 11 on the basis of the trigger information. Specifically, when receiving the trigger information from the processing unit 21, the calculation unit 32 stores, in the storage unit, the information on the output value of the biological sensor 11 transmitted from the processing unit 21.

In step ST29, the calculation unit 32 calculates the amount of the measurement object on the basis of the output value of the biological sensor 11. Specifically, the calculation unit 32 calculates the amount of the measurement object on the basis of the output value of the biological sensor 11 stored in the storage unit.

In the fourth embodiment, the calculation unit 32 calculates the moisture amount on the basis of the information on the frequency stored in the storage unit.

The calculation unit 32 transmits the information on the calculated amount of the measurement object to the operation display unit 31. The operation display unit 31 receives and displays the information on the amount of the measurement object.

In this manner, by performing steps ST21 to ST29, the intraoral measurement system 50 can calculate the amount of the measurement object.

The intraoral measurement system 50 according to the fourth embodiment can acquire the following effects.

The intraoral measurement system 50 includes the intraoral measurement device 1D having the contact surface 10a coming into contact with the measurement site in an oral cavity, and the processing device 40 that communicates with the intraoral measurement device 1D. The intraoral measurement device 1D includes the biological sensor 11, the contact detection unit 12, the processing unit 21, and the first communication unit 33. The biological sensor 11 is arranged on the contact surface 10a, and has the detection surface 11a that acquires biological information. The contact detection unit 12 acquires contact information that indicates the degree of contact between the measurement site and the contact surface 10a. The processing unit 21 outputs trigger information for starting processing of calculating the amount of the measurement object on the basis of the output value of the biological sensor 11 and the output value of the contact detection unit 12. The first communication unit 33 transmits the trigger information and the information on the output value of the biological sensor 11 to the processing device 40. The processing device 40 includes the second communication unit 41 and the operation display unit 31 and the calculation unit 32. The second communication unit 41 receives the trigger information and the information on the output value of the biological sensor 11 from the first communication unit 33 of the intraoral measurement device 1D. The calculation unit 32 calculates the amount of the measurement object on the basis of the trigger information and the information on the output value of the biological sensor 11.

In this manner, in the intraoral measurement system 50, the intraoral measurement device 1D outputs the trigger information on the basis of the output value of the biological sensor 11 and the output value of the contact detection unit 12. The processing device 40 calculates the amount of the measurement object on the basis of the trigger information and the information on the output value of the biological sensor 11 from the intraoral measurement device 1D. With such a configuration, the contact to the extent that the measurement accuracy can be guaranteed can be detected, so that the measurement accuracy of the intraoral measurement system 50 can be improved. Further, the contact between the measurement site in the oral cavity and the contact surface 10a can be easily detected.

Note that, in the fourth embodiment, an example has been described in which the processing device 40 is provided with the operation display unit 31, but the present disclosure is not limited thereto. In the processing device 40, the operation display unit 31 is an optional component. For example, the operation display unit 31 may be provided in the intraoral measurement device 1D. Alternatively, the operation display unit 31 may be provided in another external device.

In the fourth embodiment, an example has been described in which the intraoral measurement system 50 uses moisture as a measurement object, but the present disclosure is not limited thereto. It is sufficient that the intraoral measurement system 50 can measure the amount of the measurement object in an oral cavity.

In the fourth embodiment, an example has been described in which the intraoral measurement system 50 includes the intraoral measurement device 1D, but the present disclosure is not limited thereto. The intraoral measurement system 50 may use the intraoral measurement device of the second embodiment or embodiments 5 to 9 described later.

### (Fifth Embodiment)

An intraoral measurement device according to a fifth embodiment of the present disclosure will be described. Note that, in the fifth embodiment, points different from the first embodiment will be mainly described. In the fifth embodiment, the same or equivalent configurations as those in the first embodiment will be described with the same reference numerals. In addition, in the fifth embodiment, descriptions overlapping with those in the first embodiment are omitted.

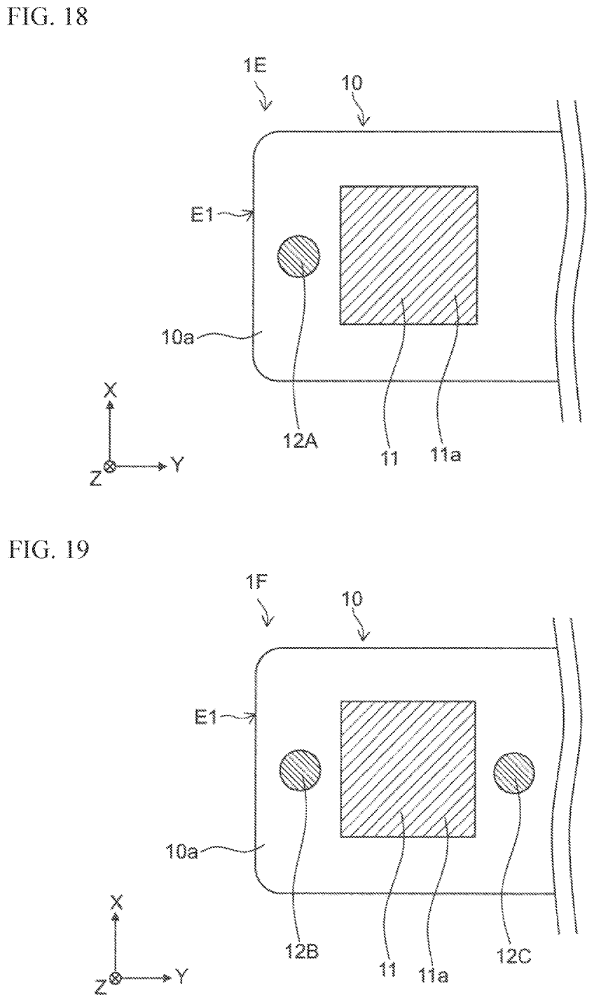

An example of the intraoral measurement device according to the fifth embodiment will be described with reference to FIG. 18. FIG. 18 is a schematic enlarged view of an example of an intraoral measurement device 1E of the fifth embodiment according to the present disclosure.

The fifth embodiment is different from the first embodiment in the position of a contact detection unit 12A.

As illustrated in FIG. 18, in the intraoral measurement device 1E, the contact detection unit 12A is arranged at a position closer to the one end E1 in the longitudinal direction D1 of the housing 2 than the biological sensor 11. In other words, the contact detection unit 12A is arranged on the one end E1 side in the longitudinal direction D1 of the housing 2 with respect to the biological sensor 11. Note that the contact detection unit 12A has substantially the same configuration as the contact detection unit 12 in the first embodiment.

In addition, the contact detection unit 12A is arranged at the center in the width direction (X direction) of the intraoral measurement device 1E as viewed in the height direction (Z direction) of the intraoral measurement device 1E.

The intraoral measurement device 1E according to the fifth embodiment can acquire the following effects.

In the intraoral measurement device 1E, the contact detection unit 12A is arranged at a position closer to the one end E1 in the longitudinal direction D1 of the housing 2 than the biological sensor 11. With such a configuration, the contact between the measurement site and the contact surface 10a can be detected with high accuracy. The one end E1 side of the housing 2 is a position that is difficult to visually check. Accordingly, by arranging the contact detection unit 12A on the one end E1 side with respect to the biological sensor 11, the contact between the measurement site and the contact surface 10a can be more easily detected with high accuracy.

Note that, in the fifth embodiment, an example has been described in which one contact detection unit 12A is arranged at a position closer to the one end E1 in the longitudinal direction D1 of the housing 2 than the biological sensor 11, but the present disclosure is not limited thereto. One or a plurality of contact detection units 12A may be arranged at positions closer to the one end E1 in the longitudinal direction D1 of the housing 2 than the biological sensor 11. By arranging a plurality of contact detection units 12A, the contact between the measurement site and the contact surface 10a can be further easily detected with high accuracy.

In the fifth embodiment, an example has been described in which the contact detection unit 12A is arranged at the center in the width direction (X direction) of the intraoral measurement device 1E as viewed in the height direction (Z direction) of the intraoral measurement device 1E, but the present disclosure is not limited thereto. It is sufficient that the contact detection unit 12A is arranged at the periphery of the biological sensor 11 on the one end E1 side of the housing 2.

### (Sixth Embodiment)

An intraoral measurement device according to a sixth embodiment of the present disclosure will be described. Note that, in the sixth embodiment, points different from the first embodiment will be mainly described. In the sixth embodiment, the same or equivalent configurations as those in the first embodiment will be described with the same reference numerals. In addition, in the sixth embodiment, descriptions overlapping with those in the first embodiment are omitted.

An example of the intraoral measurement device according to the sixth embodiment will be described with reference to FIG. 19. FIG. 19 is a schematic enlarged view of an example of an intraoral measurement device 1F of the sixth embodiment according to the present disclosure.

The sixth embodiment is different from the first embodiment in that a plurality of contact detection units 12B and 12C are provided.

As illustrated in FIG. 19, the intraoral measurement device 1F includes the plurality of contact detection units 12B and 12C. The plurality of contact detection units 12B and 12C include a first contact detection unit 12B and a second contact detection unit 12C. The first contact detection unit 12B is substantially the same as the contact detection unit 12A in the fifth embodiment, and the second contact detection unit 12C is substantially the same as the contact detection unit 12 in the first embodiment.

The first contact detection unit 12B is arranged at a position closer to the one end E1 in the longitudinal direction D1 of the housing 2 than the biological sensor 11. In other words, the first contact detection unit 12B is arranged on the one end E1 side in the longitudinal direction D1 of the housing 2 with respect to the biological sensor 11.

The second contact detection unit 12C is arranged at a position farther from the one end E1 in the longitudinal direction D1 of the housing 2 than the biological sensor 11. In other words, the second contact detection unit 12C is arranged on the center C1 side in the longitudinal direction D1 of the housing 2 with respect to the biological sensor 11.

The first contact detection unit 12B and the second contact detection unit 12C are arranged at the center in the width direction (X direction) of the intraoral measurement device 1F as viewed in the height direction (Z direction) of the intraoral measurement device 1F.

The first contact detection unit 12B and the second contact detection unit 12C face each other with the biological sensor 11 interposed therebetween. Specifically, the first contact detection unit 12B and the second contact detection unit 12C are arranged at the periphery of the biological sensor 11 symmetrically about the biological sensor 11. For example, in a case where the detection surface 11a of the biological sensor 11 has a rectangular shape, a point where two diagonals of the detection surface 11a intersect is the center of the biological sensor 11. The first contact detection unit 12B and the second contact detection unit 12C are arranged symmetrically with respect to the center of the biological sensor 11. In a case where the detection surface 11a of the biological sensor 11 has a circular shape, the first contact detection unit 12B and the second contact detection unit 12C are arranged symmetrically with respect to the center of the detection surface 11a.

The intraoral measurement device 1F according to the sixth embodiment can acquire the following effects.

The intraoral measurement device 1F includes the plurality of contact detection units 12B and 12C. The plurality of contact detection units 12B and 12C include the first contact detection unit 12B and the second contact detection unit 12C. The first contact detection unit 12B is arranged at a position closer to the one end E1 in the longitudinal direction D1 of the housing 2 than the biological sensor 11. The second contact detection unit 12C is arranged at a position farther from the one end E1 in the longitudinal direction D1 of the housing 2 than the biological sensor 11. In this manner, the first contact detection unit 12B and the second contact detection unit 12C detect the contact between the measurement site and the contact surface 10a. Accordingly, the contact between the detection surface 11a and the measurement site can be easily detected with high accuracy. As a result, the measurement accuracy of the intraoral measurement device 1F can be improved.

The plurality of contact detection units 12B and 12C are arranged at the periphery of the biological sensor 11 symmetrically about the biological sensor 11. With such a configuration, the measurement accuracy of the intraoral measurement device 1F can be further improved.

Note that, in the sixth embodiment, an example has been described in which the intraoral measurement device 1F includes one first contact detection unit 12B and one second contact detection unit 12C, but the present disclosure is not limited thereto. The intraoral measurement device 1F may include one or a plurality of first contact detection units 12B and one or a plurality of second contact detection units 12C.

In the sixth embodiment, an example has been described in which the first contact detection unit 12B and the second contact detection unit 12C are arranged symmetrically about the biological sensor 11, but the present disclosure is not limited thereto. The first contact detection unit 12B and the second contact detection unit 12C may not be arranged symmetrically about the biological sensor 11.

In the sixth embodiment, an example has been described in which the first contact detection unit 12B and the second contact detection unit 12C are arranged at the center in the width direction (X direction) of the intraoral measurement device 1F as viewed in the height direction (Z direction) of the intraoral measurement device 1F, but the present disclosure is not limited thereto.

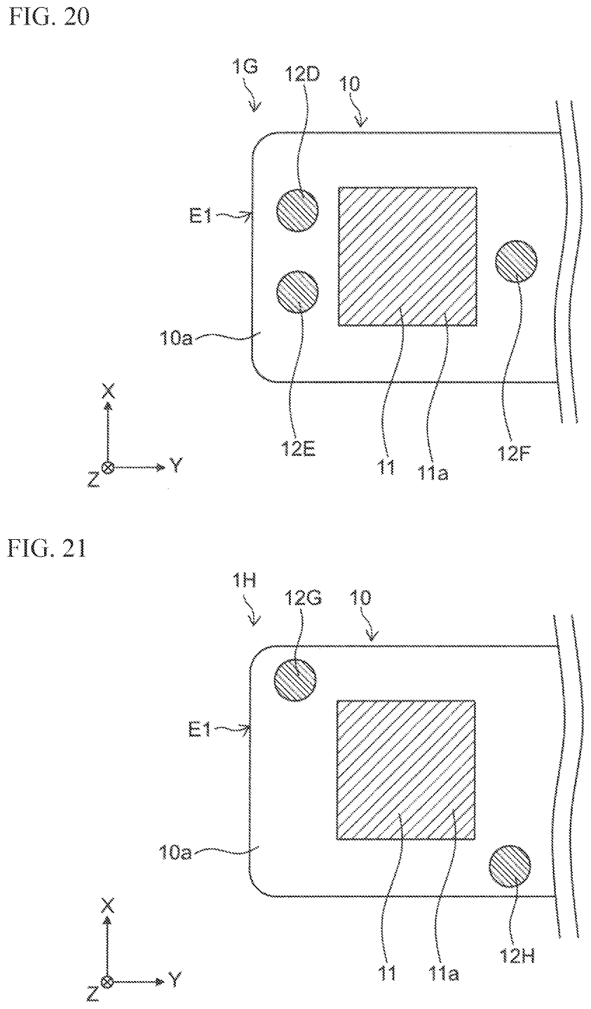

FIG. 20 is a schematic enlarged view of an intraoral measurement device 1G of a modification of the sixth embodiment according to the present disclosure. As illustrated in FIG. 20, the intraoral measurement device 1G includes three contact detection units 12D, 12E, and 12F.

In the intraoral measurement device 1G, two contact detection units 12D and 12E are arranged on the one end E1 side of the housing 2 with respect to the biological sensor 11, and one contact detection unit 12F is arranged on the center C1 side of the housing 2 with respect to the biological sensor 11.

The three contact detection units 12D, 12E, and 12F include a first contact detection unit 12D, a second contact detection unit 12E, and a third contact detection unit 12F. The first contact detection unit 12D, the second contact detection unit 12E, and the third contact detection unit 12F have substantially the same configurations as those of the contact detection unit 12 in the first embodiment.

The first contact detection unit 12D and the second contact detection unit 12E are arranged at positions closer to the one end E1 in the longitudinal direction D1 of the housing 2 than the biological sensor 11. The first contact detection unit 12D and the second contact detection unit 12E are arranged side by side along the outer peripheral portion of the detection surface 11a on the one end E1 side of the housing 2. Specifically, the first contact detection unit 12D and the second contact detection unit 12E are arranged in the X direction at the outer peripheral portion of the detection surface 11a on the one end E1 side of the housing 2.

The third contact detection unit 12F is arranged at a position farther from the one end E1 in the longitudinal direction D1 of the housing 2 than the biological sensor 11.

Also with such a configuration, by detecting the contact between the measurement site and the contact surface 10a, the contact between the detection surface 11a and the measurement site can be easily detected with high accuracy. As a result, the measurement accuracy of the intraoral measurement device 1G can be improved.

### (Seventh Embodiment)

An intraoral measurement device according to a seventh embodiment of the present disclosure will be described. Note that, in the seventh embodiment, points different from the sixth embodiment will be mainly described. In the seventh embodiment, the same or equivalent configurations as those in the sixth embodiment will be described with the same reference numerals. In addition, in the seventh embodiment, descriptions overlapping with those in the sixth embodiment are omitted.

An example of the intraoral measurement device according to the seventh embodiment will be described with reference to FIG. 21. FIG. 21 is a schematic enlarged view of an example of an intraoral measurement device 1H of the seventh embodiment according to the present disclosure.

The seventh embodiment is different from the sixth embodiment in that a plurality of contact detection units 12G and 12H are arranged at corners of the detection surface 11a.

As illustrated in FIG. 21, the intraoral measurement device 1H includes the plurality of contact detection units 12G and 12H. The plurality of contact detection units 12G and 12H are arranged at corners of the detection surface 11a of the biological sensor 11. In this specification, “arranged at corners of the detection surface 11a” includes being arranged with a predetermined distance from the corners of the detection surface 11a.

The plurality of contact detection units 12G and 12H include a first contact detection unit 12G and a second contact detection unit 12H. The first contact detection unit 12G is arranged at a corner of the detection surface 11a at a position closer to the one end E1 in the longitudinal direction D1 of the housing 2 than the biological sensor 11. The second contact detection unit 12H is arranged at a corner of the detection surface 11a at a position farther from the one end E1 in the longitudinal direction D1 of the housing 2 than the biological sensor 11. The corners of the detection surface 11a are portions where two sides that define the outer periphery of the detection surface 11a intersect when the intraoral measurement device 1H is viewed in the height direction (Z direction).

The first contact detection unit 12G and the second contact detection unit 12H are arranged symmetrically about the biological sensor 11. Specifically, the first contact detection unit 12G and the second contact detection unit 12H are arranged at two corners facing each other among four corners of the detection surface 11a having a rectangular shape.

The intraoral measurement device 1H according to the seventh embodiment can acquire the following effects.

The intraoral measurement device 1H includes the plurality of contact detection units 12G and 12H. The plurality of contact detection units 12G and 12H are arranged at corners of the detection surface 11a of the biological sensor 11. With such a configuration, the contact between the contact surface 10a and the measurement site can be easily detected with high accuracy. As a result, the measurement accuracy of the intraoral measurement device 1H can be improved.

The plurality of contact detection units 12G and 12H are arranged symmetrically about the biological sensor 11. With such a configuration, the measurement accuracy of the intraoral measurement device 1H can be further improved.

Note that, in the seventh embodiment, an example has been described in which the detection surface 11a has a rectangular shape, but the present disclosure is not limited thereto. It is sufficient that the detection surface 11a has a shape having corners. For example, the detection surface 11a may have a polygonal shape.

In the seventh embodiment, an example has been described in which the first contact detection unit 12G and the second contact detection unit 12H are arranged symmetrically about the biological sensor 11, but the present disclosure is not limited thereto. It is sufficient that the first contact detection unit 12G and the second contact detection unit 12H are arranged at corners of the detection surface 11a.

In the seventh embodiment, an example has been described in which the intraoral measurement device 1H includes two contact detection units 12G and 12H, but the present disclosure is not limited thereto. It is sufficient that the intraoral measurement device 1H includes one or a plurality of contact detection units arranged at corners of the detection surface 11a.

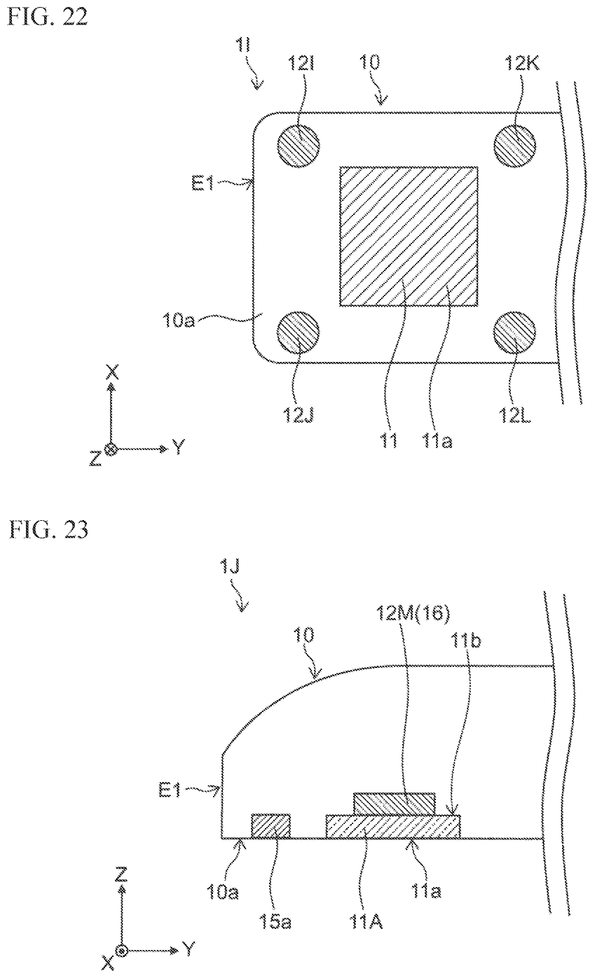

FIG. 22 is a schematic enlarged view of an intraoral measurement device 1I of a modification of the seventh embodiment according to the present disclosure. As illustrated in FIG. 22, the intraoral measurement device 1I includes four contact detection units 12I, 12J, 12K, and 12L. The four contact detection units 12I, 12J, 12K, and 12L are arranged at respective four corners of the detection surface 11a having a rectangular shape. With such a configuration, the contact between the contact surface 10a and the measurement site can be easily detected with high accuracy. As a result, the measurement accuracy of the intraoral measurement device 1I can be further improved.

### (Eighth Embodiment)

An intraoral measurement device according to an eighth embodiment of the present disclosure will be described. Note that, in the eighth embodiment, points different from the first embodiment will be mainly described. In the eighth embodiment, the same or equivalent configurations as those in the first embodiment will be described with the same reference numerals. In addition, in the eighth embodiment, descriptions overlapping with those in the first embodiment are omitted.

An example of the intraoral measurement device according to the eighth embodiment will be described with reference to FIG. 23. FIG. 23 is a schematic enlarged view of an example of an intraoral measurement device 1J of the eighth embodiment according to the present disclosure.

The eighth embodiment is different from the first embodiment in that a biological sensor 11A has translucency, a contact detection unit 12M is arranged on the biological sensor 11A, and a light emitting unit 15a is arranged on the contact surface 10a.

As illustrated in FIG. 23, in the intraoral measurement device 1J, the biological sensor 11A has translucency. For example, the biological sensor 11A is formed of a glass substrate. The biological sensor 11A has the detection surface 11a exposed to the contact surface 10a side and an arrangement surface 11b on a side opposite to the detection surface 11a.

The contact detection unit 12M is arranged on the biological sensor 11A. Specifically, the contact detection unit 12M is housed inside the sensor unit 10 of the housing 2. The contact detection unit 12M is arranged on the arrangement surface 11b of the biological sensor 11A.

The contact detection unit 12M is the optical sensor 13 that receives light. The optical sensor 13 includes the light receiving unit 16 that receives light reflected by a measurement site. The light receiving surface of the light receiving unit 16 is in contact with the arrangement surface 11b of the biological sensor 11A.

In the eighth embodiment, the contact detection unit 12M uses the biological sensor 11A having translucency as the light guide unit 14 in the first embodiment. That is, the biological sensor 11A is provided on the contact surface 10a, and guides light to the light receiving unit 16 of the contact detection unit 12M.

In the intraoral measurement device 1J, the light emitting unit 15a is arranged on the contact surface 10a. The light emitting unit 15a emits light. The light emitted from the light emitting unit 15a is reflected by the measurement site, passes through the biological sensor 11A, and enters the light receiving unit 16 of the contact detection unit 12M.

In this manner, the light receiving unit 16 of the contact detection unit 12M receives the light emitted from the light emitting unit 15a arranged on the contact surface 10a through the biological sensor 11A having translucency.

The intraoral measurement device 1J according to the eighth embodiment can acquire the following effects.

In the intraoral measurement device 1J, the biological sensor 11A has translucency, and has the arrangement surface 11b on a side opposite to the detection surface 11a. The contact detection unit 12M includes the optical sensor 13 that receives light, and is arranged on the arrangement surface 11b of the biological sensor 11A. Such a configuration can reduce the number of components, so that manufacturing cost can be reduced, and miniaturization can be realized.

Note that, in the eighth embodiment, an example has been described in which the intraoral measurement device 1J includes one contact detection unit 12M, but the present disclosure is not limited thereto. The intraoral measurement device 1J may include one or a plurality of contact detection units 12M arranged on the arrangement surface 11b of the biological sensor 11A. In addition, the intraoral measurement device 1J may include the contact detection unit 12 in the first embodiment at the periphery of the biological sensor 11A.

In the eighth embodiment, an example has been described in which the light emitting unit 15a is arranged on the contact surface 10a, but the present disclosure is not limited thereto. For example, the light emitting unit 15a may be housed inside the sensor unit 10 of the housing 2, and be arranged on the arrangement surface 11b of the biological sensor 11A.

### (Ninth Embodiment)

An intraoral measurement device according to a ninth embodiment of the present disclosure will be described. Note that, in the ninth embodiment, points different from the first embodiment will be mainly described. In the ninth embodiment, the same or equivalent configurations as those in the first embodiment will be described with the same reference numerals. In addition, in the ninth embodiment, descriptions overlapping with those in the first embodiment are omitted.

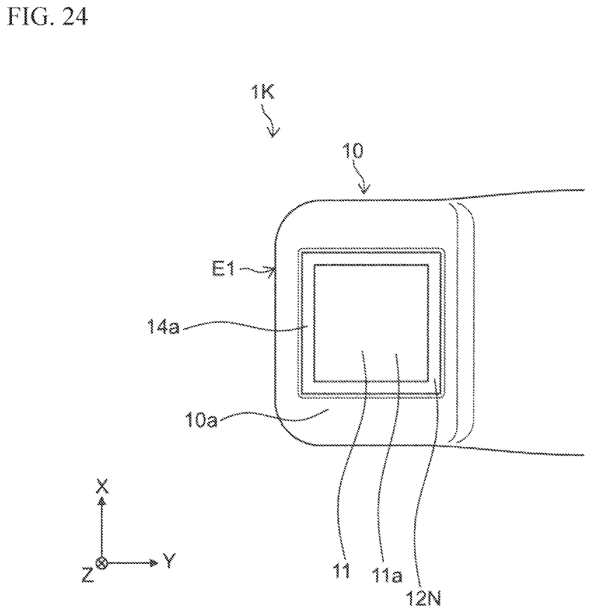

An example of the intraoral measurement device according to the ninth embodiment will be described with reference to FIG. 24. FIG. 24 is a schematic enlarged view of an example of an intraoral measurement device 1K of the ninth embodiment according to the present disclosure.

The ninth embodiment is different from the first embodiment in that a contact detection unit 12N surrounds the periphery of the detection surface 11a of the biological sensor 11.

As illustrated in FIG. 24, in the intraoral measurement device 1K, the contact detection unit 12N surrounds the periphery of the detection surface 11a of the biological sensor 11. The contact detection unit 12N includes the optical sensor 13 and a light guide unit 14a that guides light to the optical sensor 13.

The optical sensor 13 is housed inside the sensor unit 10 of the housing 2. The light guide unit 14a has a frame shape and covers the outer periphery of the detection surface 11a having a rectangular shape. The light guide unit 14a is, for example, formed of a light guide plate.

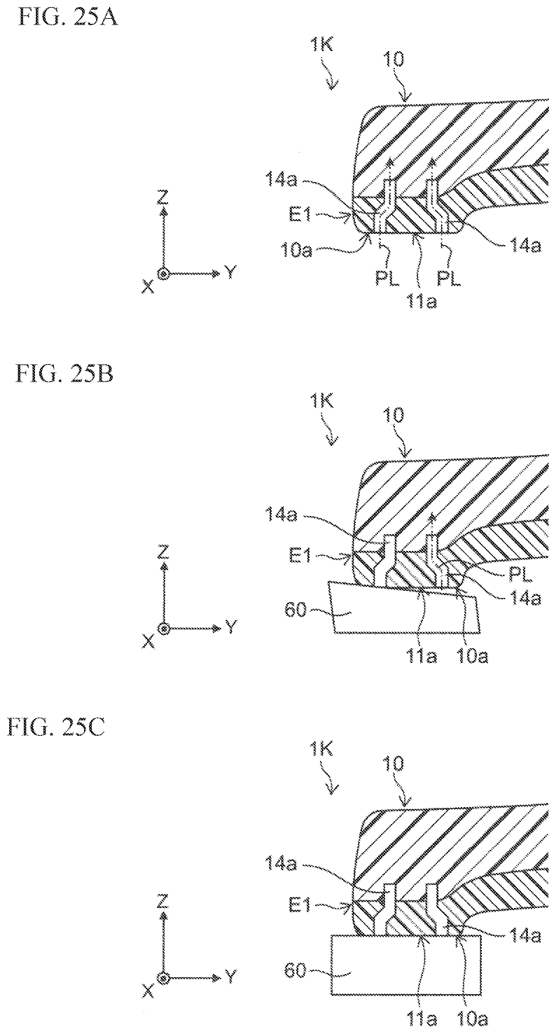

FIGS. 25A to 25C are schematic views of examples of operations of the intraoral measurement device 1K of the ninth embodiment according to the present disclosure. FIG. 25A illustrates an operation in a state in which the contact surface 10a is not in contact with a measurement site 60. FIG. 25B illustrates an operation in a state in which the contact surface 10a is obliquely in contact with the measurement site 60. FIG. 25C illustrates an operation in a state in which the contact surface 10a is in surface contact with the measurement site 60.

As illustrated in FIG. 25A, in the case where the contact surface 10a is not in contact with the measurement site 60, light PL enters the light guide unit 14a. The light PL entered the light guide unit 14a is received by the light receiving unit 16 of the optical sensor 13.

As illustrated in FIG. 25B, in the case where the contact surface 10a is obliquely in contact with the measurement site 60, the light PL is blocked by the measurement site 60 at a portion of the light guide unit 14a that is in contact with the measurement site 60. On the other hand, the light PL enters a portion of the light guide unit 14a that is not in contact with the measurement site 60.

As illustrated in FIG. 25C, in the case where the contact surface 10a is in surface contact with the measurement site 60, the light PL to enter the light guide unit 14a is blocked.

In this manner, by surrounding the periphery of the detection surface 11a of the biological sensor 11 with the light guide unit 14a of the contact detection unit 12N, the contact between the measurement site 60 and the contact surface 10a can be detected with higher accuracy.

The intraoral measurement device 1K according to the ninth embodiment can acquire the following effects.

In the intraoral measurement device 1K, the contact detection unit 12N surrounds the periphery of the detection surface 11a of the biological sensor 11. With such a configuration, the contact between the measurement site 60 and the contact surface 10a can be detected with higher accuracy, so that the measurement accuracy can be improved.

Note that, in the ninth embodiment, an example has been described in which the contact detection unit 12N includes one light guide unit 14a having a frame shape, but the present disclosure is not limited thereto. For example, the contact detection unit 12N may include one or a plurality of light guide units. In a case where the contact detection unit 12N includes a plurality of light guide units, the plurality of light guide units may be arranged at the outer periphery of the detection surface 11a.

Although the present disclosure has been fully described in connection with embodiments with reference to the accompanying drawings, various modifications and corrections are apparent to those skilled in the art. Such modifications and corrections should be understood to be included within the scope of the present disclosure according to the appended claims as long as they do not depart therefrom.

### INDUSTRIAL APPLICABILITY

The intraoral measurement device and the intraoral measurement system of the present disclosure can be applied to, for example, a moisture amount measurement device for measuring a moisture amount in an oral cavity.

### DESCRIPTION OF REFERENCE SYMBOLS

1A, 1B, 1C, 1D, 1E, 1F, 1G, 1H, 1I, 1J, 1K: Intraoral measurement device

2: Housing

10: Sensor unit

11, 11A: Biological sensor

11a: Detection surface

11b: Arrangement surface

12, 12A, 12B, 12C, 12D, 12E, 12F, 12G, 12H, 121, 12J, 12K, 12L, 12M, 12N: Contact detection unit

13: Optical sensor

14, 14a: Light guide unit

15, 15a: Light emitting unit

16: Light receiving unit

17: Recess

20: Probe unit

21: Processing unit

30: Grip unit

31: Operation display unit

32: Calculation unit

33: First communication unit

40: Processing device

41: Second communication unit

50: Intraoral measurement system

60: Measurement site

1. An intraoral measurement device having a contact surface configured to contact a measurement site in an oral cavity, the device comprising:
a biological sensor that is on the contact surface, and that has a detection surface configured to acquire biological information; and
at least one contact detection unit sensor that is on the biological sensor or at a periphery of the biological sensor, and that is configured to acquire contact information indicating a degree of contact between the measurement site and the contact surface.

2. The intraoral measurement device according to claim 1, further comprising:
a housing that has a rod shape and that houses the biological sensor and the contact detection unit sensors,
wherein the contact surface is at a first end side along a longitudinal direction of the housing.

3. The intraoral measurement device according to claim 2, wherein the at least one contact detection unit sensor is farther from the first end of the housing than a center of the biological sensor.

4. The intraoral measurement device according to claim 2, wherein the at least one contact detection unit sensor is closer to the first end of the housing than a center of the biological sensor.

5. The intraoral measurement device according to claim 2, comprising a plurality of contact detection unit sensors, wherein the plurality of contact detection unit sensors comprise:
at least one first contact detection unit sensor closer to the first end of the housing than a center of the biological sensor; and
at least one second contact detection unit sensor farther from the first end of the housing than the center of the biological sensor.

6. The intraoral measurement device according to claim 1, comprising a plurality of contact detection unit sensors, wherein:
the detection surface of the biological sensor has a polygonal shape, and
the plurality of contact detection unit sensors are arranged at corners of the detection surface.

7. The intraoral measurement device according to claim 5, comprising a plurality of contact detection unit sensors, wherein the plurality of contact detection unit sensors are arranged symmetrically about the biological sensor.

8. The intraoral measurement device according to claim 1, comprising a plurality of contact detection unit sensors, wherein the plurality of contact detection unit sensors surround a periphery of the detection surface of the biological sensor.

9. The intraoral measurement device according to claim 1, comprising a plurality of contact detection unit sensors, wherein the plurality of contact detection unit sensors comprise:
at least one optical sensor configured to receive light; and
at least one light guide on the contact surface and configured to guide the received light to the optical sensors.

10. The intraoral measurement device according to claim 9, wherein each of the optical sensors comprises:
a light emitter configured to emit light; and
a light receiver configured to receive light reflected by the measurement site.

11. The intraoral measurement device according to claim 1, comprising a plurality of contact detection unit sensors, wherein:
the biological sensor is translucent and has an arrangement surface on a side opposite to the detection surface, and
the plurality of contact detection unit sensors comprise at least one optical sensor configured to receive light and arranged on the arrangement surface of the biological sensor.

12. The intraoral measurement device according to claim 11, further comprising a light emitter that is arranged on the contact surface and that is configured to emit light.

13. The intraoral measurement device according to claim 1, wherein the biological sensor is a capacitive sensor configured to detect a capacitance.

14. The intraoral measurement device according to claim 1, further comprising a processor configured to output trigger information that starts a determination of an amount of a measurement object based on outputs of the biological sensor and the at least one contact detection unit sensor.

15. The intraoral measurement device according to claim 14, wherein the processor is configured to determine the amount of the measurement object by using the output of the biological sensor after outputting the trigger information.

16. The intraoral measurement device according to claim 1, further comprising:
a display having a main surface configured to display the biological information,
wherein the contact surface and the main surface of the display are parallel to each other.

17. The intraoral measurement device according to claim 1, further comprising:
a display having a main surface configured to display the biological information,
wherein the contact surface and the main surface of the display face opposite directions.

18. The intraoral measurement device according to claim 1, further comprising:
a substrate having a main surface on which a processor is mounted, the processor being configured to control the at least one contact detection unit sensor,
wherein the contact surface and the main surface of the substrate are parallel to each other.
